(19) United States
(12) Patent Application Publication
(10) Pub. No.: US 2026/0263455 A1
(43) Pub. Date: Sep. 10, 2026

(54) FORMULATIONS OF AN ANTIVIRAL PRODRUG

(71) Applicant: Exavir Therapeutics, Inc., San Francisco, CA (US)

(72) Inventors: Gary T. ELLIOTT, San Francisco, CA (US); James ANDERSON, San Francisco, CA (US); Jianmin XU, San Francisco, CA (US); Janet GUNZNER-TOSTE, San Francisco, CA (US); Alborz YAZDI, San Francisco, CA (US)

(21) Appl. No.: 19/165,963

(22) PCT Filed: Mar. 13, 2024
(86) PCT No.: PCT/US2024/019703

§ 371 Date: Sep. 16, 2025

Related U.S. Application Data
(60) Provisional application No. 63/491,002, filed on Mar. 17, 2023.

(51) Int. Cl. A61K 31/4985; A61K 9/14; A61K 47/26; A61P 31/18

(52) U.S. Cl. CPC A61K 31/4985; A61K 9/14; A61K 47/26; A61P 31/18

(57) ABSTRACT

Disclosed herein, in part, are pharmaceutical compositions comprising a prodrug of an antiviral agent and methods of using the same in the treatment of viral infections.

### CROSS-REFERENCE TO RELATED APPLICATIONS

This application claims the benefit of priority to U.S. Provisional Patent Application No. 63/491,002, filed Mar. 17, 2023, which application is hereby incorporated by reference in its entirety.

### SUMMARY

Disclosed herein, in certain embodiments, is a pharmaceutical composition comprising an antiviral prodrug and a cryoprotectant.

Described herein, in certain embodiments, is a pharmaceutical composition (e.g., an aqueous suspension) comprising: (i) a crystalline form of a compound represented by:

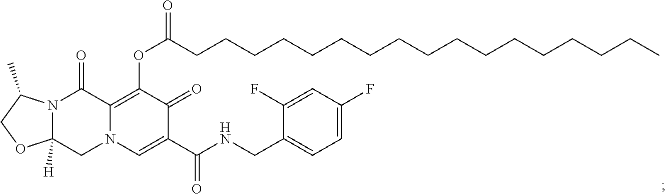 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant.

In some embodiments, the cryoprotectant is a sugar. In some embodiments, the cryoprotectant is selected from the group consisting of trehalose, sucrose, mannitol, and any combination thereof. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the pharmaceutical composition comprises a phosphate buffer. In some embodiments, the pH of the composition is from about 6.0 to about 7.5. In some embodiments, the pH of the composition is about 7.0. In some embodiments, the pharmaceutical composition comprises about 25 weight percent to about 45 weight percent of the crystalline form, based on the total weight of the pharmaceutical composition. In some embodiments, the pharmaceutical composition comprises a plurality of nanoparticles comprising the crystalline form of the compound. In some embodiments, the pharmaceutical composition comprises a plurality of nanoparticles comprising the crystalline form of the compound and the surfactant. In some embodiments, the pharmaceutical composition comprises a plurality of nanoparticles comprising the crystalline form of the compound and the polyethylene glycol. In some embodiments, the pharmaceutical composition comprises a plurality of nanoparticles comprising the crystalline form of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles a polydispersity index of about 0.25 to about 0.35.

Described herein, in certain embodiments, is a pharmaceutical composition (e.g., an aqueous suspension) comprising: (i) a mixture of crystalline forms of a compound represented by:

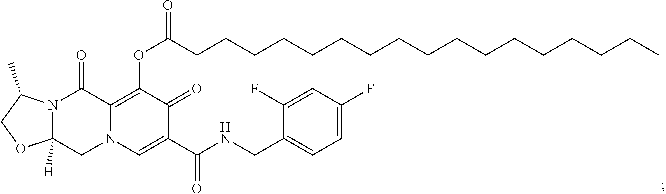 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant.

In some embodiments, the cryoprotectant is a sugar. In some embodiments, the cryoprotectant is selected from the group consisting of trehalose, sucrose, mannitol, and any combination thereof. In some embodiments, the cryoprotectant is selected from the group consisting of trehalose, sucrose, and mannitol. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the pharmaceutical composition comprises a phosphate buffer. In some embodiments, the pH of the composition is from about 6.0 to about 7.5. In some embodiments, the pH of the composition is about 7.0. In some embodiments, the mixture of crystalline forms of the compound has two crystalline forms. In some embodiments, one of the two crystalline forms is present in about 10 to 30 weight percent of the mixture and the other crystalline form in about 70 to 90 weight percent of the mixture. In some embodiments, one of the two crystalline forms is present in about 15 to 30 weight percent of the mixture and the other crystalline form in about 70 to 85 weight percent of the mixture. In some embodiments, the mixture of crystalline forms of the compound is characterized by a powder X-ray diffraction pattern having a characteristic peak in degrees 2θ at about 21.6. In some embodiments, the mixture of crystalline forms of the compound is characterized by a powder X-ray diffraction pattern having a characteristic peak in degrees 2θ at about 7.0, 17.8, and 21.6. In some embodiments, the mixture of crystalline forms of the compound is characterized by a powder X-ray diffraction pattern having a characteristic peak in degrees 2θ at about 7.0, 7.4, 17.8, 21.6, and 23.6. In some embodiments, the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1. In some embodiments, the mixture of crystalline forms of the compound is characterized by an XRPD pattern substantially as depicted in FIG. 2. In some embodiments, the pharmaceutical composition comprises about 25 weight percent to about 45 weight percent of the mixture of crystalline forms of the compound, based on the total weight of the pharmaceutical composition. In some embodiments, the pharmaceutical composition comprises a plurality of nanoparticles comprising the mixture of crystalline forms of the compound. In some embodiments, the pharmaceutical composition comprises a plurality of nanoparticles comprising the mixture of crystalline forms of the compound and the surfactant. In some embodiments, the pharmaceutical composition comprises a plurality of nanoparticles comprising the mixture of crystalline forms of the compound and the polyethylene glycol. In some embodiments, the pharmaceutical composition comprises a plurality of nanoparticles comprising the mixture of crystalline forms of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles has a polydispersity index of about 0.25 to about 0.35.

Described herein, in certain embodiments, is a method of treating, inhibiting, and/or preventing a viral infection in a patient in need thereof, comprising administering to the patient an effective amount of a pharmaceutical composition described herein.

In some embodiments, the viral infection is a retroviral infection. In some embodiments, the viral infection is an HIV infection. In some embodiments, the method comprises administering the pharmaceutical composition intramuscularly.

Described herein, in certain embodiments, is a process for preparing a pharmaceutical composition comprising: (i) a crystalline form of a compound represented by:

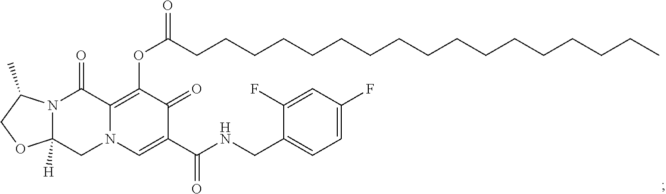 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant; the process comprising: (a) providing a suspension comprising the crystalline form of the compound; (b) mixing the suspension using a ball milling rotor, thereby preparing a ball-milled mixture; and (c) combining the ball-milled mixture with the cryoprotectant, thereby preparing the pharmaceutical composition.

In some embodiments, the suspension comprises one or more excipients selected from the group consisting of a polyethylene glycol, a surfactant, and combinations thereof. In some embodiments, the cryoprotectant is a sugar. In some embodiments, the cryoprotectant is selected from the group consisting of trehalose, sucrose, mannitol, and any combination thereof. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the suspension further comprises a surfactant. In some embodiments, the pharmaceutical composition comprises about 3 weight percent to about 5 weight percent of the surfactant, based on the total weight of the pharmaceutical composition. In some embodiments, the suspension further comprises a polyethylene glycol. In some embodiments, the pharmaceutical composition comprises about 3 weight percent to about 5 weight percent of the polyethylene glycol, based on the total weight of the pharmaceutical composition. In some embodiments, the process comprises combining the ball-milled mixture with the cryoprotectant with a phosphate buffer. In some embodiments, the pH of the composition is from about 6.0 to about 7.5. In some embodiments, the pH of the composition is about 7.0. In some embodiments, the pharmaceutical composition comprises about 25 weight percent to about 45 weight percent of the crystalline form, based on the total weight of the pharmaceutical composition. In some embodiments, the pharmaceutical composition comprises a plurality of nanoparticles comprising the crystalline form of the compound. In some embodiments, the pharmaceutical composition comprises a plurality of nanoparticles comprising the crystalline form of the compound and the surfactant. In some embodiments, the pharmaceutical composition comprises a plurality of nanoparticles comprising the crystalline form of the compound and the polyethylene glycol. In some embodiments, the pharmaceutical composition comprises a plurality of nanoparticles comprising the crystalline form of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles in the pharmaceutical composition has a polydispersity index of about 0.25 to about 0.35.

Described herein, in certain embodiments, process for preparing a pharmaceutical composition comprising: (i) a mixture of crystalline forms of a compound represented by:

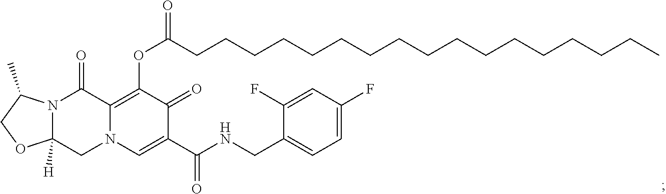 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant; the process comprising: (a) providing a suspension comprising the mixture of crystalline forms of the compound; (b) mixing the suspension using a ball milling rotor, thereby preparing a ball-milled mixture; and (c) combining the ball-milled mixture with the cryoprotectant, thereby preparing the pharmaceutical composition.

In some embodiments, the cryoprotectant is a sugar. In some embodiments, the cryoprotectant is selected from the group consisting of trehalose, sucrose, mannitol, and any combination thereof. In some embodiments, the cryoprotectant is selected from the group consisting of trehalose, sucrose, and mannitol. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the the pharmaceutical composition comprises a phosphate buffer. In some embodiments, the pH of the composition is from about 6.0 to about 7.5. In some embodiments, the pH of the composition is about 7.0. In some embodiments, the mixture of crystalline forms of the compound has two crystalline forms. In some embodiments, one of the two crystalline forms is present in about 10 to 30 weight percent of the mixture and the other crystalline form is about 70 to 90 weight percent of the mixture. In some embodiments, the mixture of crystalline forms of the compound is characterized by a powder X-ray diffraction pattern having a characteristic peak in degrees 2θ at about 21.6. In some embodiments, the mixture of crystalline forms of the compound is characterized by a powder X-ray diffraction pattern having a characteristic peak in degrees 2θ at about 7.0, 17.8, and 21.6. In some embodiments, the mixture of crystalline forms of the compound is characterized by a powder X-ray diffraction pattern having a characteristic peak in degrees 2θ at about 7.0, 7.4, 17.8, 21.6, and 23.6. In some embodiments, the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1. In some embodiments, the mixture of crystalline forms of the compound is characterized by an XRPD pattern substantially as depicted in FIG. 2. In some embodiments, the pharmaceutical composition comprises about 25 weight percent to about 45 weight percent of the mixture of crystalline forms of the compound, based on the total weight of the pharmaceutical composition. In some embodiments, the pharmaceutical composition comprises a plurality of nanoparticles comprising the mixture of crystalline forms of the compound. In some embodiments, the pharmaceutical composition comprises a plurality of nanoparticles comprising the mixture of crystalline forms of the compound and the surfactant. In some embodiments, the pharmaceutical composition comprises a plurality of nanoparticles comprising the mixture of crystalline forms of the compound and the polyethylene glycol. In some embodiments, the pharmaceutical composition comprises a plurality of nanoparticles comprising the mixture of crystalline forms of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, plurality of nanoparticles has a z-average particle size of 275 nm to 425 nm. In some embodiments, the plurality of nanoparticles has a polydispersity index of about 0.15 to about 0.4. In some embodiments, the plurality of nanoparticles has a polydispersity index of about 0.25 to about 0.35.

### BRIEF DESCRIPTION OF THE DRAWINGS

FIG. 1 depicts a histogram of particle size distribution of Formulation F16 after exposure to gamma radiation.

FIG. 2 depicts an exemplary XRPD pattern of a mixture of crystalline forms of M2CAB in pharmaceutical compositions of the disclosure (Form 1+4).

### DETAILED DESCRIPTION

### Definitions

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

As used herein, “pharmaceutically acceptable excipient” refers to any substance in a pharmaceutical formulation other than an active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

As used herein, “M2CAB” refers to the compound having the structure:

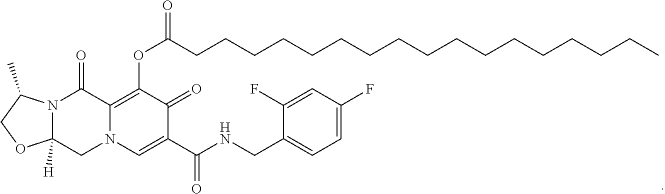 [SMILES: CCCCCCCCCCCCCCCCCC(=O)Oc1c2n(cc(C(=O)NCc3ccc(F)cc3F)c1=O)C[C@H]1OC[C@H](C)N1C2=O]

### Pharmaceutical Compositions

Disclosed herein, in some embodiments, are pharmaceutical compositions comprising: (a) a crystalline solid form of M2CAB, and (b) a pharmaceutically acceptable excipient.

Described herein, in certain embodiments, is a pharmaceutical composition (e.g., an aqueous suspension) comprising: (i) a crystalline form of a compound represented by:

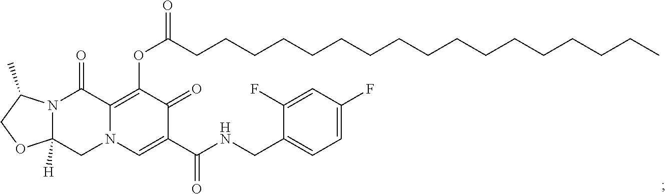 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant.

In some embodiments, the cryoprotectant is a sugar. In some embodiments, the cryoprotectant is selected from the group consisting of trehalose, sucrose, mannitol, and any combination thereof. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the pharmaceutical composition comprises a phosphate buffer. In some embodiments, the pH of the composition is from about 6.0 to about 7.5. In some embodiments, the pH of the composition is about 7.0. In some embodiments, the pharmaceutical composition comprises about 25 weight percent to about 45 weight percent of the crystalline form, based on the total weight of the pharmaceutical composition. In some embodiments, the pharmaceutical composition comprises a plurality of nanoparticles comprising the crystalline form of the compound. In some embodiments, the pharmaceutical composition comprises a plurality of nanoparticles comprising the crystalline form of the compound and the surfactant. In some embodiments, the pharmaceutical composition comprises a plurality of nanoparticles comprising the crystalline form of the compound and the polyethylene glycol. In some embodiments, the pharmaceutical composition comprises a plurality of nanoparticles comprising the crystalline form of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles a polydispersity index of about 0.25 to about 0.35.

Described herein, in certain embodiments, is a pharmaceutical composition comprising: (i) a mixture of crystalline forms of a compound represented by:

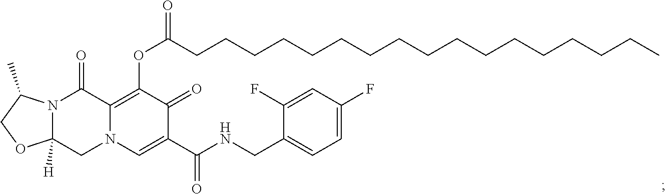 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant.

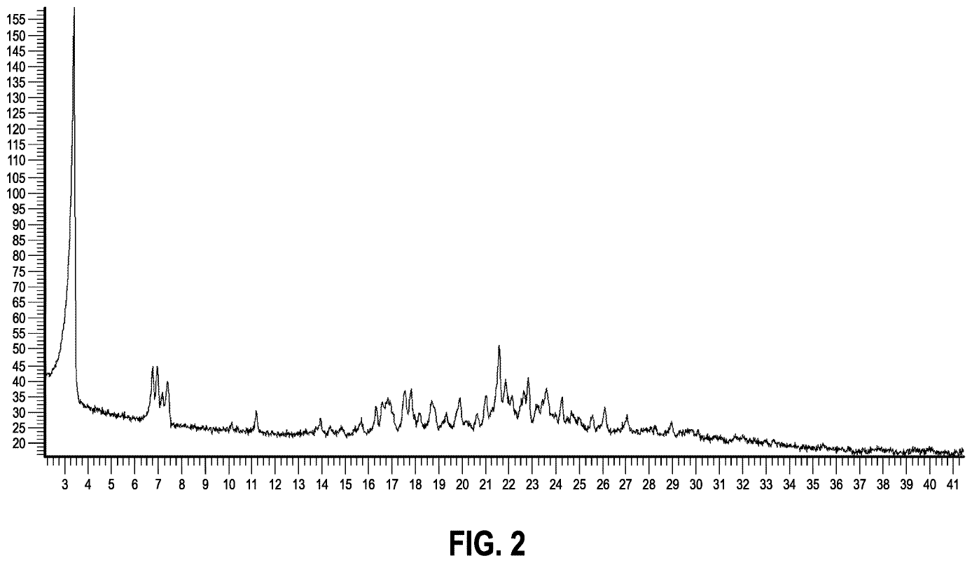

In some embodiments, the cryoprotectant is a sugar. In some embodiments, the cryoprotectant is selected from the group consisting of trehalose, sucrose, mannitol, and any combination thereof. In some embodiments, the cryoprotectant is selected from the group consisting of trehalose, sucrose, and mannitol. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the pharmaceutical composition comprises a phosphate buffer. In some embodiments, the pH of the composition is from about 6.0 to about 7.5. In some embodiments, the pH of the composition is about 7.0. In some embodiments, the mixture of crystalline forms of the compound has two crystalline forms. In some embodiments, one of the two crystalline forms is present in about 10 to 30 weight percent of the mixture and the other crystalline form in about 70 to 90 weight percent of the mixture. In some embodiments, one of the two crystalline forms is present in about 15 to 30 weight percent of the mixture and the other crystalline form in about 70 to 85 weight percent of the mixture. In some embodiments, the mixture of crystalline forms of the compound is characterized by a powder X-ray diffraction pattern having a characteristic peak in degrees 2θ at about 21.6. In some embodiments, the mixture of crystalline forms of the compound is characterized by a powder X-ray diffraction pattern having a characteristic peak in degrees 2θ at about 7.0, 17.8, and 21.6. In some embodiments, the mixture of crystalline forms of the compound is characterized by a powder X-ray diffraction pattern having a characteristic peak in degrees 2θ at about 7.0, 7.4, 17.8, 21.6, and 23.6. In some embodiments, the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1. In some embodiments, the mixture of crystalline forms of the compound is characterized by an XRPD pattern substantially as depicted in FIG. 2. In some embodiments, the pharmaceutical composition comprises about 25 weight percent to about 45 weight percent of the mixture of crystalline forms of the compound, based on the total weight of the pharmaceutical composition. In some embodiments, the pharmaceutical composition comprises a plurality of nanoparticles comprising the mixture of crystalline forms of the compound. In some embodiments, the pharmaceutical composition comprises a plurality of nanoparticles comprising the mixture of crystalline forms of the compound and the surfactant. In some embodiments, the pharmaceutical composition comprises a plurality of nanoparticles comprising the mixture of crystalline forms of the compound and the polyethylene glycol. In some embodiments, the pharmaceutical composition comprises a plurality of nanoparticles comprising the mixture of crystalline forms of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles has a polydispersity index of about 0.25 to about 0.35.

In some embodiments, the pharmaceutical composition is an aqueous suspension.

In some embodiments, the pharmaceutical composition comprises about 3 weight percent to about 10 weight percent of the cryoprotectant, based on the total weight of the pharmaceutical composition. In some embodiments, the pharmaceutical composition comprises about 3 weight percent of the cryoprotectant, based on the total weight of the pharmaceutical composition. In some embodiments, the pharmaceutical composition comprises about 4 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises about 6 weight percent of the cryoprotectant, based on the total weight of the pharmaceutical composition. In some embodiments, the pharmaceutical composition comprises about 7 weight percent of the cryoprotectant, based on the total weight of the pharmaceutical composition. In some embodiments, the pharmaceutical composition comprises about 8 weight percent of the cryoprotectant, based on the total weight of the pharmaceutical composition. In some embodiments, the pharmaceutical composition comprises about 9 weight percent of the cryoprotectant, based on the total weight of the pharmaceutical composition. In some embodiments, the pharmaceutical composition comprises about 10 weight percent of the cryoprotectant, based on the total weight of the pharmaceutical composition.

In some embodiments, the pharmaceutical composition comprises a surfactant. In some embodiments, the pharmaceutical composition comprises about 1 weight percent to about 10 weight percent of the surfactant, based on the total weight of the pharmaceutical composition. In some embodiments, the pharmaceutical composition comprises about 3 weight percent to about 5 weight percent of the surfactant, based on the total weight of the pharmaceutical composition. In some embodiments, the pharmaceutical composition comprises about 3 weight percent of the surfactant, based on the total weight of the pharmaceutical composition. In some embodiments, the pharmaceutical composition comprises about 4 weight percent of the surfactant, based on the total weight of the pharmaceutical composition. In some embodiments, the pharmaceutical composition comprises about 5 weight percent of the surfactant, based on the total weight of the pharmaceutical composition.

In some embodiments, the pharmaceutical composition comprises a polyethylene glycol. In some embodiments, the pharmaceutical composition comprises about 1 weight percent to about 10 weight percent of the polyethylene glycol, based on the total weight of the pharmaceutical composition. In some embodiments, the pharmaceutical composition comprises about 3 weight percent to about 5 weight percent of the polyethylene glycol, based on the total weight of the pharmaceutical composition. In some embodiments, the pharmaceutical composition comprises about 3 weight percent of the polyethylene glycol, based on the total weight of the pharmaceutical composition. In some embodiments, the pharmaceutical composition comprises about 4 weight percent of the polyethylene glycol, based on the total weight of the pharmaceutical composition. In some embodiments, the pharmaceutical composition comprises about 5 weight percent of the polyethylene glycol, based on the total weight of the pharmaceutical composition.

In some embodiments, the pharmaceutical composition comprises about 10 to about 45 weight percent of the crystalline form of the compound, based on the total weight of the pharmaceutical composition. In some embodiments, the pharmaceutical composition comprises about 25 to about 45 weight percent of the crystalline form of the compound, based on the total weight of the pharmaceutical composition. In some embodiments, the pharmaceutical composition comprises about 25 to about 40 weight percent of the crystalline form of the compound, based on the total weight of the pharmaceutical composition. In some embodiments, the pharmaceutical composition comprises about 25 to about 35 weight percent of the crystalline form of the compound, based on the total weight of the pharmaceutical composition. In some embodiments, the pharmaceutical composition comprises about 25 to about 30 weight percent of the crystalline form of the compound. In some embodiments, the pharmaceutical composition comprises about 30 to about 45 weight percent of the crystalline form of the compound, based on the total weight of the pharmaceutical composition. In some embodiments, the pharmaceutical composition comprises about 30 to about 40 weight percent of the crystalline form of the compound, based on the total weight of the pharmaceutical composition. In some embodiments, the pharmaceutical composition comprises about 35 to about 45 weight percent of the crystalline form of the compound, based on the total weight of the pharmaceutical composition. In some embodiments, the pharmaceutical composition comprises about 25 weight percent of the crystalline form of the compound, based on the total weight of the pharmaceutical composition. In some embodiments, the pharmaceutical composition comprises about 30 weight percent of the crystalline form of the compound, based on the total weight of the pharmaceutical composition. In some embodiments, the pharmaceutical composition comprises about 35 weight percent of the crystalline form of the compound, based on the total weight of the pharmaceutical composition. In some embodiments, the pharmaceutical composition comprises about 40 weight percent of the crystalline form of the compound, based on the total weight of the pharmaceutical composition. In some embodiments, the pharmaceutical composition comprises about 45 weight percent of the crystalline form of the compound, based on the total weight of the pharmaceutical composition.

In some embodiments, the composition has a physiologically compatible pH (e.g., a range from a pH of about 3 to a pH of about 11, about pH 3 to about pH 7, depending on the formulation and route of administration). In some cases, the pH is from about pH 5.0 to about pH 8.

A pharmaceutical composition disclosed herein is administered by any appropriate route that results in effective treatment in the subject. In some embodiments, a pharmaceutical composition disclosed herein is administered systemically. In some embodiments, a pharmaceutical composition disclosed herein is administered locally. The pharmaceutical composition is administered via a route such as, but not limited to, enteral, gastroenteral, oral, transdermal, subcutaneous, nasal, intravenous, intravenous bolus, intravenous drip, intraarterial, intramuscular, transmucosal, insufflation, sublingual, buccal, conjunctival, cutaneous. Modes of administration include injection, infusion, instillation, and/or ingestion. “Injection” includes, without limitation, intravenous, intramuscular, intraarterial, intrathecal, intraventricular, intradermal, intraperitoneal, transtracheal, and subcutaneous. In some embodiments, the route is intramuscular. In some embodiments, the route is subcutaneously.

### Methods of Use

Disclosed herein, in some embodiments, are methods of treating an HIV-1 infection in an individual in need thereof. Further disclosed herein, in some embodiments, are methods of preventing an HIV-1 infection in an individual in need thereof. Additionally, disclosed herein, in some embodiments, are methods of preventing transmission of an HIV-1 virus from one individual to another (for example, from a pregnant woman to a child, for example during birth or breast feeding).

Described herein, in certain embodiments, is a method of treating, inhibiting, and/or preventing a viral infection in an individual in need thereof, comprising administering to the individual an effective amount of a pharmaceutical composition described herein.

Disclosed herein, in certain embodiments, are methods of treating, inhibiting, and/or preventing a viral infection in an individual in need thereof, comprising administering a pharmaceutical composition (e.g., a pharmaceutical composition described herein) comprising an effective amount of a crystalline form of a compound represented by:

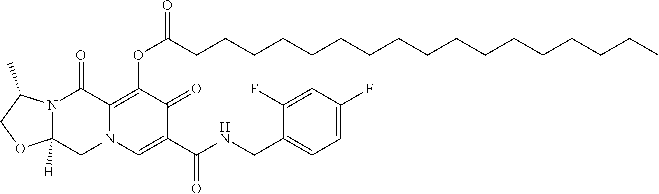 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual.

In some embodiments, the viral infection is a retroviral infection. In some embodiments, the viral infection is an HIV infection. In some embodiments, the method comprises administering the pharmaceutical composition intramuscularly to the individual. In some embodiments, the method comprises administering the pharmaceutical composition subcutaneously to the individual.

In some embodiments, the method comprises administering the mixture of the crystalline form of the compound to the individual once monthly. In some embodiments, the method comprises administering the mixture of the crystalline form of the compound to the individual once every two months. In some embodiments, the method comprises administering the mixture of the crystalline form of the compound to the individual once every three months. In some embodiments, the method comprises administering the mixture of the crystalline form of the compound to the individual once every six months. In some embodiments, the method comprises administering the mixture of the crystalline form of the compound to the individual once every nine months. In some embodiments, the method comprises administering the mixture of the crystalline form of the compound of the compound to the individual once every twelve months.

Disclosed herein, in certain embodiments, are methods of treating an HIV-1 infection in an individual in need thereof, comprising administering a pharmaceutical composition (e.g., a pharmaceutical composition described herein) comprising a therapeutically effective amount of a crystalline form of a compound represented by:

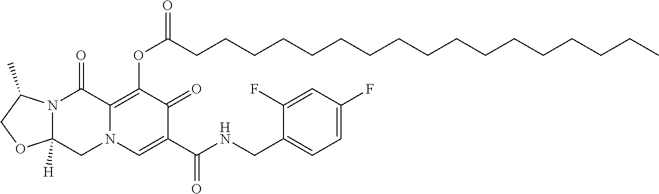 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual. Further disclosed herein, in some embodiments, are methods of preventing an HIV-1 infection in an individual in need thereof, comprising administering a pharmaceutical composition (e.g., a pharmaceutical composition described herein) comprising a prophylactically effective amount of crystalline form of a compound represented by:

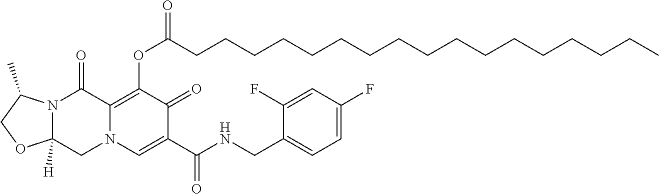 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual. Additionally, disclosed herein, in some embodiments, are methods of preventing transmission of an HIV-1 virus from one individual to another (for example, from a pregnant woman to a child, for example during birth or breast feeding), comprising administering a pharmaceutical composition (e.g., a pharmaceutical composition described herein) comprising a prophylactically effective amount of a crystalline form of a compound represented by:

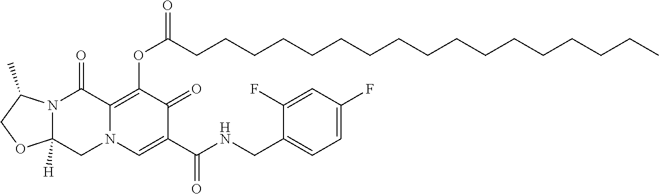 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual.

Disclosed herein, in certain embodiments, are methods of treating an HIV-1 infection in an individual in need thereof, comprising administering a pharmaceutical composition (e.g., a pharmaceutical composition described herein) comprising a therapeutically effective amount of a mixture of crystalline forms of a compound represented by:

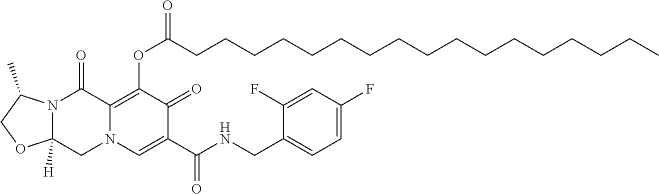 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual. Further disclosed herein, in some embodiments, are methods of preventing an HIV-1 infection in an individual in need thereof, comprising administering a pharmaceutical composition (e.g., a pharmaceutical composition described herein) comprising a prophylactically effective amount of a mixture of crystalline forms represented by:

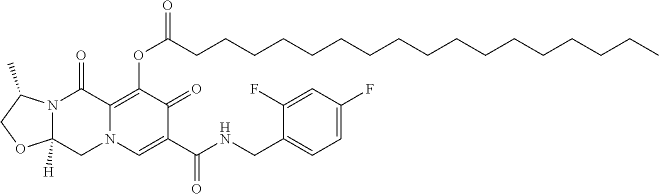 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual. Additionally, disclosed herein, in some embodiments, are methods of preventing transmission of an HIV-1 virus from one individual to another (for example, from a pregnant woman to a child, for example during birth or breast feeding), comprising administering a pharmaceutical composition (e.g., a pharmaceutical composition described herein) comprising a prophylactically effective amount of mixture of crystalline forms of a compound represented by:

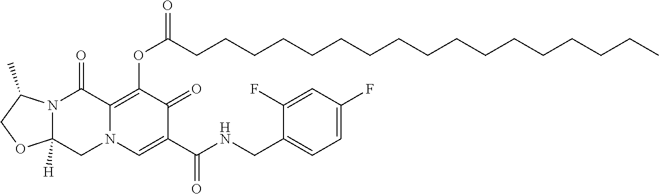 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and a pharmaceutically acceptable excipient to the individual.

In some embodiments, the viral infection is a retroviral infection. In some embodiments, the viral infection is an HIV infection. In some embodiments, the method comprises administering the pharmaceutical composition intramuscularly to the individual. In some embodiments, the method comprises administering the pharmaceutical composition subcutaneously to the individual.

In some embodiments, the method comprises administering the mixture of the crystalline form of the compound to the individual once monthly. In some embodiments, the method comprises administering the mixture of the crystalline form of the compound to the individual once every two months. In some embodiments, the method comprises administering the mixture of the crystalline form of the compound to the individual once every three months. In some embodiments, the method comprises administering the mixture of the crystalline form of the compound to the individual once every six months. In some embodiments, the method comprises administering the mixture of the crystalline form of the compound to the individual once every nine months. In some embodiments, the method comprises administering the mixture of the crystalline form of the compound of the compound to the individual once every twelve months.

In some embodiments, crystalline forms of a M2CAB described herein, mixtures of crystalline forms of M2CAB described herein, or pharmaceutical compositions described herein, are administered in combination with an additional agent to treat a disorder described herein.

### Methods of Making

Described herein, in certain embodiments, is a process for preparing a pharmaceutical composition comprising: (i) a crystalline form of a compound represented by:

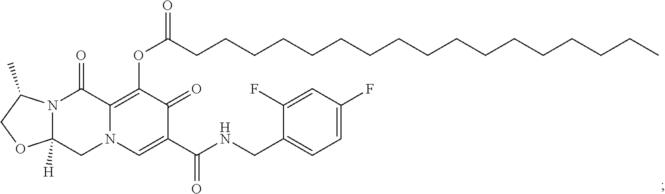 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant; the process comprising: (a) providing a suspension comprising the crystalline form of the compound; (b) mixing the suspension using a ball milling rotor, thereby preparing a ball-milled mixture; and (c) combining the ball-milled mixture with the cryoprotectant, thereby preparing the pharmaceutical composition.

In some embodiments, the suspension comprises a polyethylene glycol. In some embodiments, the suspension comprises a surfactant. In some embodiments, the suspension comprises one or more excipients selected from the group consisting of a polyethylene glycol, a surfactant, and combinations thereof. In some embodiments, the cryoprotectant is a sugar. In some embodiments, the cryoprotectant is selected from the group consisting of trehalose, sucrose, mannitol, and any combination thereof. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the suspension further comprises a surfactant. In some embodiments, the pharmaceutical composition comprises about 3 weight percent to about 5 weight percent of the surfactant, based on the total weight of the pharmaceutical composition. In some embodiments, the suspension further comprises a polyethylene glycol. In some embodiments, the pharmaceutical composition comprises about 3 weight percent to about 5 weight percent of the polyethylene glycol, based on the total weight of the pharmaceutical composition. In some embodiments, the process comprises combining the ball-milled mixture with the cryoprotectant with a phosphate buffer. In some embodiments, the pH of the composition is from about 6.0 to about 7.5. In some embodiments, the pH of the composition is about 7.0. In some embodiments, the pharmaceutical composition comprises about 25 weight percent to about 45 weight percent of the crystalline form, based on the total weight of the pharmaceutical composition. In some embodiments, the pharmaceutical composition comprises a plurality of nanoparticles comprising the crystalline form of the compound. In some embodiments, the pharmaceutical composition comprises a plurality of nanoparticles comprising the crystalline form of the compound and the surfactant. In some embodiments, the pharmaceutical composition comprises a plurality of nanoparticles comprising the crystalline form of the compound and the polyethylene glycol. In some embodiments, the pharmaceutical composition comprises a plurality of nanoparticles comprising the crystalline form of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, the plurality of nanoparticles has a z-average particle size of 275 nm to 425 nm. In some embodiments, the plurality of nanoparticles has a polydispersity index of about 0.15 to about 0.4. In some embodiments, the plurality of nanoparticles in the pharmaceutical composition has a polydispersity index of about 0.25 to about 0.35.

Described herein, in certain embodiments, process for preparing a pharmaceutical composition comprising: (i) a mixture of crystalline forms of a compound represented by:

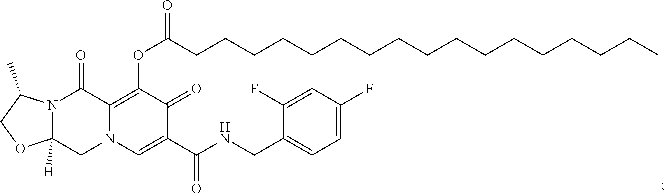 [SMILES: CCCCCCCCCCCCCCCCCC(=O)Oc1c2n(cc(C(=O)NCc3ccc(F)cc3F)c1=O)C[C@H]1OC[C@H](C)N1C2=O]

and (ii) a cryoprotectant; the process comprising: (a) providing a suspension comprising the mixture of crystalline forms of the compound; (b) mixing the suspension using a ball milling rotor, thereby preparing a ball-milled mixture; and (c) combining the ball-milled mixture with the cryoprotectant, thereby preparing the pharmaceutical composition.

In some embodiments, the suspension comprises a polyethylene glycol. In some embodiments, the suspension comprises a surfactant. In some embodiments, the cryoprotectant is a sugar. In some embodiments, the cryoprotectant is selected from the group consisting of trehalose, sucrose, mannitol, and any combination thereof. In some embodiments, the cryoprotectant is selected from the group consisting of trehalose, sucrose, and mannitol. In some embodiments, the cryoprotectant is trehalose. In some embodiments, the pharmaceutical composition comprises about 3 weight percent to about 10 weight percent of the cryoprotectant, based on the total weight of the pharmaceutical composition. In some embodiments, the pharmaceutical composition comprises about 5 weight percent of the cryoprotectant, based on the total weight of the pharmaceutical composition. In some embodiments, the pharmaceutical composition comprises a surfactant. In some embodiments, the pharmaceutical composition comprises about 3 weight percent to about 5 weight percent of the surfactant, based on the total weight of the pharmaceutical composition. In some embodiments, the pharmaceutical composition comprises a polyethylene glycol. In some embodiments, the pharmaceutical composition comprises about 3 weight percent to about 5 weight percent of the polyethylene glycol, based on the total weight of the pharmaceutical composition. In some embodiments, the pharmaceutical composition comprises a phosphate buffer. In some embodiments, the pH of the composition is from about 6.0 to about 7.5. In some embodiments, the pH of the composition is about 7.0. In some embodiments, the mixture of crystalline forms of the compound has two crystalline forms. In some embodiments, one of the two crystalline forms is present in about 10 to 30 weight percent of the mixture and the other crystalline form is about 70 to 90 weight percent of the mixture. In some embodiments, the mixture of crystalline forms of the compound is characterized by a powder X-ray diffraction pattern having a characteristic peak in degrees 2θ at about 21.6. In some embodiments, the mixture of crystalline forms of the compound is characterized by a powder X-ray diffraction pattern having a characteristic peak in degrees 2θ at about 7.0, 17.8, and 21.6. In some embodiments, the mixture of crystalline forms of the compound is characterized by a powder X-ray diffraction pattern having a characteristic peak in degrees 2θ at about 7.0, 7.4, 17.8, 21.6, and 23.6. In some embodiments, the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1. In some embodiments, the mixture of crystalline forms of the compound is characterized by an XRPD pattern substantially as depicted in FIG. 2. In some embodiments, the pharmaceutical composition comprises about 25 weight percent to about 45 weight percent of the mixture of crystalline forms of the compound, based on the total weight of the pharmaceutical composition. In some embodiments, the pharmaceutical composition comprises a plurality of nanoparticles comprising the mixture of crystalline forms of the compound. In some embodiments, the pharmaceutical composition comprises a plurality of nanoparticles comprising the mixture of crystalline forms of the compound and the surfactant. In some embodiments, the pharmaceutical composition comprises a plurality of nanoparticles comprising the mixture of crystalline forms of the compound and the polyethylene glycol. In some embodiments, the pharmaceutical composition comprises a plurality of nanoparticles comprising the mixture of crystalline forms of the compound, the surfactant, and the polyethylene glycol. In some embodiments, the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm. In some embodiments, the plurality of nanoparticles has a z-average particle size of 200 nm to 900 nm. In some embodiments, plurality of nanoparticles has a z-average particle size of 275 nm to 425 nm. In some embodiments, the plurality of nanoparticles has a polydispersity index of about 0.15 to about 0.4. In some embodiments, the plurality of nanoparticles has a polydispersity index of about 0.25 to about 0.35.

### EXAMPLES

Abbreviations: API: active pharmaceutical ingredient; DP: drug product; DS: drug substance; NT: not tested; ND: not determined PSD: particle size distribution; QS: quantum satis (quantity sufficient).

### Example 1. DP Process to Make M2CAB Drug Product; F1 Formulation

TABLE 1.1
Materials and Supplies

| Name | Grade |
|---|---|
| M2CAB (Prodrug) | N/A |
| Tween 20 | N.F |
| PEG-3350 | USP |
| Sodium Chloride | USP |
| Potassium Chloride | USP |
| Potassium Phosphate | HPLC |
| Monobasic | |
| Sodium Hydroxide | N/A |

TABLE 1.2
Instruments
Name

Analytical balance
Balance
pH meter
Mini Bead beater
Shear-Jet (high pressure
homogenizer)
Zetasizer

TABLE 1.3
Composition of F1 Formulation (w/v %)

| Materials | F1 (%) |
|---|---|
| M2CAB (Prodrug) | 45 |
| Tween 20 | 6.43 |
| PEG-3350 | 6.43 |
| PBS (pH 7.0) | QS |

TABLE 1.4
Compounding of F1 Formulation (10 mL Batch)

| Materials | F1 (mg) |
|---|---|
| M2CAB (Prodrug) | 4500 |
| Tween 20 | 643 |
| PEG-3350 | 643 |
| PBS (pH 7.0) | 10 mL |

### Procedure

Preparation of PBS: To approximately 790 g of deionized water, approximately 1.6 g of potassium phosphate monobasic, 8 g of sodium chloride and 2 g of potassium chloride was added in a glass bottle. Following dissolution, the pH of the solution was measured and adjusted to pH 7 by adding sodium hydroxide. The solution was quantity sufficient to 1000 g by adding deionized water.

### Preparation of Formulation

Approximately 645 mg of Tween 20 (polyoxyethylene (20) sorbitan monolaurate) was added into a 20-mL glass vial via pipette, followed by addition of 642 mg of PEG-3350 polymer and 10 mL of PBS (pH 7.0). The solution was slowly mixed at 300 rpm (to avoid bubbles) for 20 minutes using a magnetic stir bar until the polymer was completely dissolved. Approximately 4500 mg of M2CAB powder was transferred portion-wise into the glass vial. The mixture was slowly mixed at 700 revolutions per minute (rpm) using a magnetic stir bar until the M2CAB was completely uniformly dispersed (about 18 hours). An in-process check by visual observation and microscopic image was performed until little to no clumps were observed. Once the M2CAB was uniformly dispersed, the bead beating process below was commenced.

0.25 mL of 0.4 mm zirconium oxide beads and 1 mL of formulation was added into a microtube. Shaking at 4800 rpm for 2 minutes was commenced (initial temperature: 23° C.; final temperature: 36° C.) with one-minute pulses after 1 min. After bead beating, the suspension was loaded into a pre-set and cleaned with a high shear homogenizer (5-30 psi), passing the suspension through the high-pressure chamber until the particle size (Z-avg) was no more than 400 nm. In process checks by an analyzer were performed. Once the particle size (Z-avg) was less than 400 nm, the samples were collected in 20-mL glass vials and appearance, pH, and osmolality were tested and recorded in the tables below.

TABLE 1.5
Particle size distribution of F1 after bead beating

| Time | Z-avg(nm) | PDI | D10 (i) (nm) | D50(i) (nm) | D90(i) (nm) |
|---|---|---|---|---|---|
| 2 min | 624.7 | 0.123 | 529 | 611 | 713 |

TABLE 1.6
Particle size distribution of F1 after Microfluidizer

| | Z-avg | | | | |
|---|---|---|---|---|---|
| Pass | (nm) | PDI | D10 (i) (nm) | D50(i) (nm) | D90(i) (nm) |
| 1 | 363.9 | 0.192 | 218 | 406 | 705 |

TABLE 1.7
pH of F1 Formulation

| Sample Name | pH |
|---|---|
| F1 | 7.23 |

TABLE 1.8
Osmolality of F1 vehicle and Formulation

| Sample Name | Osmolality (mOsm) |
|---|---|
| F1 vehicle | 479 |
| F1 | 465 |

### Discussion

After 2 min of bead beating, Z-Avg (particle size distribution, or PSD) of F1 was reduced to 624 nm. Followed by bead beating and one pass of the high shear homogenizer, the Z-avg was 363 nm. It was noted that the F1 nanosuspension formulation was thicker after 18 hours of stirring and difficult to transfer by pipette.

### Example 2. Gamma Vs. X-Ray Irradiation of F1 Formulation and Stability Over 17 Days

Purpose: To analyze appearance, particle size distribution and Zeta-potential for the F1 formulation post gamma and X-ray irradiation and to evaluate the stability of the F1 formulation over 17 days. For the irradiation studies, the target dose was 25 kGy The actual dose delivery by gamma irradiation was between 26.8 and 27.2 kGy with a total exposure time of 519 minutes. For the X-ray irradiation, the actual dose was between 30.6 to 31.0 kGy with a total exposure time of 125 minutes.

TABLE 2.1
Preparation of F1 samples for analysis

| | | Weighed | PBS added | |
|---|---|---|---|---|
| Sample | Dilution | Sample(mg) | (mg) | Remark |
| F1 2-8° C. stability sample (17 days) | 10x | 102.3 | 912.6 | Easily diluted |
| F1 25° C. stability sample (17 days) | 100x | 10.6 | 992.6 | Difficult to |
| | | | | dissolve at 10X |
| F1 stability sample X-ray (11 days | 100x | 10.2 | 992.2 | Difficult to |
| after x-ray irradiation) | | | | dissolve at 10X |
| F1 stability sample Gamma (1 day | 100x | 10.3 | 1002.7 | Difficult to |
| after gamma irradiation) | | | | dissolve at 10X |

### Results

TABLE 2.2
Appearance of F1 Samples

| Sample | Appearance |
|---|---|
| F1 2-8° C. stability sample | White, opaque, liquidified |
| (17 days) | |
| F1 25° C. stability sample | White and cake form |
| (17 days) | |
| F1 STB X-ray (11 days | White and cake form |
| after x-rays irradiation) | |
| F1 STB Gamma (1 day | White and cake form |
| after gamma irradiation) | |

TABLE 2.3
pH of F1 Samples

| Sample | pH |
|---|---|
| F1 2-8° C. stability sample (17 days) | 7.05 |
| F1 25° C. stability sample (17 days) | 7.05 |
| F1 STB X-ray (After 11 days of x-rays irradiation) | 7.05 |
| F1 STB Gamma (After 1 day of gamma irradiation) | 7.04 |

TABLE 2.4
Particle size distribution of F1 samples

| Sample | Z-avg(nm) | PDI | D(i)10 (nm) | D(i)50(nm) | D(i)90(nm) |
|---|---|---|---|---|---|
| F1 2-8° C. stability | 360.1 | 0.249 | 191 | 391 | 895 |
| sample (17 days) | | | | | |
| F1 25° C. stability | 646.0 | 0.408 | 357 | 498 | 691 |
| sample (17 days) | | | | | |
| F1 STB X-ray (11 | 504.2 | 0.417 | 267 | 382 | 546 |
| days after x-rays | | | | | |
| irradiation) | | | | | |
| F1 STB Gamma (1 | 526.0 | 0.440 | 281 | 370 | 483 |
| day after of gamma | | | | | |
| irradiation) | | | | | |

TABLE 2.5
Zeta Potential of F1 samples

| Sample | Zeta Potential (mV) |
|---|---|
| F1 2-8° C. stability sample (17 days) | −20.5 |
| F1 25° C. stability sample (17 days) | −19.1 |
| F1 stability sample X-ray (11 days after x-rays | |
| irradiation) | −26.2 |
| F1 stability sample Gamma (1 day after gamma | −20.4 |
| irradiation) | |

### Discussion

Results showed the F1 formulation was not stable in that at 25° C., the F1 formulation became a cake form while F1 stored at 2-8° C. remained fluid. Following both gamma and X-ray treatment, the F1 formulation also developed a cake like physical form. The samples that developed the cake like form had a significant increase in the particle size distribution (PSD) as measured by D (10), D (50) and D (90).

### Example 3. M2CAB Nano-Suspension Formulation with Ball Milling

TABLE A-1
Materials:

| Name | Grade |
|---|---|
| M2CAB (Prodrug) | N/A |
| Tween 20 | N/A |
| PEG-3350 | N/A |
| Sodium Phosphate | ACS |
| Monobasic | |
| Trehalose | NF, EP, JP ChP |
| DI Water | N/A |
| HCl | N/A |
| NaOH | N/A |

TABLE A-2
F13 formulation compounding table for 200 g

| Components | Active | Weight % |
|---|---|---|
| M2CAB (Prodrug) | 74.25 | 36.1 |
| Tween 20 | 10.61 | 5.1 |
| PEG-3350 | 10.61 | 5.1 |
| Sodium phosphate monobasic, monohydrate | 0.16 | |
| 5% w/w Trehalose water solution | 110 | QS |
| Total | 205.63 | |

### Procedure

### Preparation of 5% Trehalose Solution (200 mL)

To approximately 10 g of trehalose was added. 200 g of DI water in a glass container, and mixed to form a clear solution.

### Preparation of Formulation

Approximately 10 g of Tween 20 (polyoxyethylene (20) sorbitan monolaurate) and 10 g of PEG-3350 polymer were added to a 500 mL glass bottle. 110 g of the 5% trehalose solution was transferred into a glass vial and mixed at 700 rpm for 10 minutes. until completely dissolved. The solution was then mixed using a high shear mixer at 5,000 rpm, using ice. Approximately 10 g of M2CAB powder. was transferred into the solution, and mixing resumed at 5,000 rpm for 2 minutes. The above steps were repeated and the weight transferred, mixing speed and duration were recorded in the table below:

TABLE A-3

| Intended | Actual | Mixing | | |
|---|---|---|---|---|
| wt (g) | Wt (g) | speed (rpm) | Duration | |
| 10 | 10.276 | 5,000 | 2 | minutes |
| 10 | 10.131 | 5,000 | 2 | minutes |
| 10 | 10.180 | 5,000 | 2 | minutes |
| 10 | 10.670 | 5,000 | 2 | minutes |
| 10 | 10.204 | 5,000 | 2 | minutes |
| 10 | 10.186 | 5,000 | 2 | minutes |
| 14.25 | 14.895 | 12,000 | 20 | minutes |
| Total | 76.542 | N/A | 32 | minutes |

The solution was slowly mixed using a high shear, with ice, until the M2CAB was completely uniformly dispersed.

### Ball-Milling Process:

Approximately 140-150 mL of milling beads (0.4 mm zirconium oxide beads) were added to the grinding tank of a mill using a feed funnel, and the mill moved. to the horizontal operating position. The F13 formulation was added to the collecting vessel of the mill. Overhead stiring was turned on, the cooling water cocks opened, and the product pump switched on, starting with lowest flow rate. As soon as the product exited the outlet into the collecting vessel, the ball mill was started at its lowest speed (10 Hz or 600 rpm) and increased to 2400 rpm in less than a minute. The F13 formulation was milled in the mill at 2400 rpm, gradually increasing the F13 formulation circulation rate while maintaining a pressure of no more than 10. Samples were collected at the time points in the table below and the particle size distribution (PSD) was measured.

TABLE A-4

| Time Point | D10 | D50 | D90 | | Z-Average |
|---|---|---|---|---|---|
| (minutes) | (nm) | (nm) | (nm) | PDI | (nm) |
| 5 | 299 | 545 | 1020 | 0.196 | 480.6 |
| 10 | 175 | 572 | 1220 | 0.384 | 425.6 |
| 15 | 181 | 459 | 1200 | 0.280 | 380.2 |
| 20 | 223 | 380 | 717 | 0.257 | 381.9 |
| 25 | 206 | 369 | 652 | 0.243 | 363.9 |
| 30 | 220 | 378 | 1010 | 0.262 | 387.4 |
| 35 | 200 | 403 | 1250 | 0.255 | 376.5 |

The F13 formulation was milled until the particle size (D50) reached plateau and collected into a glass bottle. Buffer agents were added and mixed by stirring or overhead mixer. pH was adjusted if needed using IN NaOH or HCl.

TABLE A-5
Final product results summary

| Criteria | F13 |
|---|---|
| Appearance | White to off-white suspension |
| Flowability | Free flowing |
| pH | 7.16 |
| PSD (nm) | 376.5 |
| PDI | 0.255 |
| HPLC assay (mg/g) | 375 |
| HPLC assay (mg/mL) | 363 |
| HPLC impurity | No new impurity peak, or |
| | peak growth observed |

### Example 4. M2CAB Nano-Suspension Formulation with Ball Milling

TABLE 9.1
Formulation With New Batch of Prodrug

| Name | Grade |
|---|---|
| M2CAB (Prodrug) | N/A |
| Tween 20 | N/A |
| PEG-3350 | N/A |
| Sodium Phosphate Monobasic | ACS |
| trehalose | NF, EP, JP ChP |
| DI water | N/A |
| HCl | N/A |
| NaOH | N/A |

TABLE 9.2
F14 Formulation Composition (% w/v)

| Components | F14 |
|---|---|
| M2CAB (Prodrug) | 5.36 |
| Tween 20 | 1 |
| PEG-3350 | 1 |
| water | QS |

TABLE 9.3
F14 formulation compounding table for 200 g

| | | |
|---|---|---|
| Components | Active | % Wt |

| | | | |
|---|---|---|---|
| M2CAB (Prodrug) | 62 | g | 30.2 |
| Tween 20 | 11.5716 | g | |
| PEG-3350 | 11.5716 | g | |
| water | 120 | mL | |

### Procedure-Preparation of Formulation

Approximately 11 g of Tween 20 (polyoxyethylene (20) sorbitan monolaurate) and 11 g of PEG-3350 polymer were transferred into a glass bottle. 119 g of water was added and the solution was slowly mixed at 700 rpm for 15 min using a magnetic stir bar until the polymer was completely dissolved. 62 g of M2CAB powder was then transferred step-wise into the glass bottle.

The solution was slowly mixed using a magnetic stir bar, at 1,200 rpm for 80 hours (high shearing was used due to uniformity issue of formulation and keep stirring after finishing high shearing) until the M2CAB was completely uniformly dispersed, then the remaining M2CAB was added step-wise into the glass bottle.

### High-Shear Process

The suspension sample was placed into an ice bath and homogenized with a Polytron high shear mixer at 1,200 rpm at 2-8° C. for 20 minutes. An in-process check by an analyzer was performed every 5 minutes (taking about 5 μL each time), and the results recorded in the table below. The process was stopped if the particle size was between 800-900 nm.

| Shearing time | | | | | |
|---|---|---|---|---|---|
| (min) | Z-avg | PDI | D10 (nm) | D50 (nm) | D90 (nm) |
| 5 | 2501 | 0.489 | 1920 | 2410 | 3020 |
| 10 | 2722 | 0.652 | 1990 | 2510 | 3230 |

### Ball-Milling Process:

140 mL of milling beads (0.4 mm zirconium oxide beads) were added to the grinding tank of the mill using a feed funnel, and the mill moved into the horizontal operating position. The F14 formulation was added to the collecting vessel of the mill, the mill turned on and the overhead stirrer turned on. The cooling water cocks were opened and the product pump switched on, starting with the lowest flow rate.

As soon as the product exited the outlet into the collecting vessel, the ball mill was started at its lowest speed (10 Hz or 600 rpm) and increased to 2400 rpm in less than a minute. The F14 formulation was milled at 2400 rpm. The F14 formulation circulation rate was gradually increased while maintaining a pressure of no more than 10, at a pump setting of 4. The samples were collected at time points in the table below, and the particle size distribution (PSD) was measured:

| Time Point | | | | | |
|---|---|---|---|---|---|
| (minutes) | Z-avg (nm) | PDI | D10 (nm) | D50 (nm) | D90 (nm) |
| 10 | 376.8 | 0.245 | 191 | 436 | 836 |
| 20 | 315.3 | 0.189 | 188 | 351 | 655 |

Milling of the F14 formulation was continued until the particle size was near about 300 nm. The ball milling sample was collected into a bottle, and the milled F14 formulations stored at 2-8° C. The appearance was observed visually and by optical microscopy, and the particle size distribution tested.

TABLE 9.4
Final product results summary

| Criteria | F14 |
|---|---|
| Appearance | White to off-white suspension |
| Flowability | Free flowing |
| pH | 8.04 |
| PSD (nm) | 315.3 |
| PDI | 0.189 |
| Density (g/mL) | 0.9705 |
| HPLC assay (mg/g) | 327.9 |
| HPLC assay (mg/mL) | 318.23 |
| HPLC impurity | No new impurity peak, |
| | or peak growth observed |

Furthermore, no clogging was observed during the process.

### Example 5. Freeze Thaw Study of F14

Cryoprotectants for the formulation F14 using particle size distribution (PSD) measurements were screened as described below.

### Procedure

### 3% Trehalose

Approximately 30 mg of trehalose and 1 mL of F14 were added into a vial. The vial and vortex well were sealed and crimped. The particle size distribution (PSD) was measured, and the vial was stored at −20° C.

### 5% Trehalose

Approximately 50 mg of trehalose and 1 mL of F14 were added into a vial. The vial and vortex well were sealed and crimped. The particle size distribution (PSD) was measured, and the vial was stored at −20° C.

### 10% Trehalose

Approximately 100 mg of trehalose and 1 mL of F14 were added into a vial. The vial and vortex well were sealed and crimped. The particle size distribution (PSD) was measured, and the vial was stored at −20° C.

### 3% Mannitol

Approximately 30 mg of mannitol and 1 mL of F14 were added into a vial. The vial and vortex well were sealed and crimped. The PSD was measured, and the vial was stored at −20° C.

### 5% Mannitol

Approximately 50 mg of mannitol and 1 mL of F14 were added into a vial. The vial and vortex well were sealed and crimped. The PSD was measured, and the vial was stored at −20° C.

### Testing

The osmolality was tested using an osmometer following the initialization procedure with a sample tube filled with 0.25 mL of DI water, as instructed. Once the machine initialized, calibration was verified by running 200 μL of a Clinitrol 290 mOsm standard. The result was recorded as 288 mOSm. Once the Osmometer reached 290 mOSm±2, the probe was cleaned with DI water and gently dried with a wipe. The sample tube was then replaced with a new one containing 200 μL of sample, and the result was recorded.

### Results

TABLE 10.1
Osmolality* (mOsm/kg) of formulations prepared

| | 3% | 5% | 10% | 3% | 5% |
|---|---|---|---|---|---|
| Name | trehalose | trehalose | trehalose | Mannitol | Mannitol |
| Reading | 112 | 135 | 237 | 147 | 269 |
| Dilution | 2 | 2 | 2 | 2 | 2 |
| factor | | | | | |
| Osmolality | 224 | 270 | 474 | 294 | 538 |

*Result is corrected with dilution factor (1:1 sample:DI water), Osmolality = Reading × Dilution factor

TABLE 10.2
Particle Size Distribution and Osmolality After Freeze-thaw

| | | Avg | | D(i)10 | D(i)50 | D(i)90 |
|---|---|---|---|---|---|---|
| Sample (%) | Cycles | (nm) | PDI | (nm) | (nm) | (nm) |
| 3% trehalose | 0 | 317.8 | 0.241 | 167 | 380 | 783 |
| | 1 | 364.7 | 0.313 | 175 | 430 | 928 |
| | 2 | 331.0 | 0.236 | 178 | 362 | 757 |
| | 3 | 323.9 | 0.223 | 171 | 377 | 794 |
| 5% trehalose | 0 | 321.0 | 0.249 | 160 | 386 | 819 |
| | 1 | 366.4 | 0.323 | 169 | 450 | 1170 |
| | 2 | 327.1 | 0.240 | 177 | 353 | 787 |
| | 3 | 333.5 | 0.241 | 170 | 406 | 825 |
| 10% trehalose | 0 | 318.2 | 0.245 | 173 | 352 | 809 |
| | 1 | 335.0 | 0.299 | 171 | 363 | 1270 |
| | 2 | 325.2 | 0.224 | 186 | 364 | 702 |
| | 3 | 342.4 | 0.274 | 193 | 364 | 836 |
| 3% Mannitol | 0 | 323.0 | 0.244 | 171 | 380 | 760 |
| | 1 | 343.3 | 0.256 | 176 | 398 | 840 |
| | 2 | 344.2 | 0.321 | 176 | 367 | 824 |
| | 3 | 355.1 | 0.449 | 156 | 405 | 1250 |
| 5% Mannitol | 0 | 326.0 | 0.247 | 163 | 398 | 819 |
| | 1 | 338.2 | 0.327 | 179 | 353 | 677 |
| | 2 | 358.5 | 0.314 | 172 | 429 | 938 |
| | 3 | 378.0 | 0.238 | 204 | 456 | 878 |

### Discussion

After 3 cycles of freeze-thaw, all three concentrations of trehalose remain the same (no significant change) in particle size.
After 3 cycles of freeze-thaw, all two concentrations of mannitol contain no significant change in particle size.

### Example 6. Freeze Thaw Study of F14

Purpose: To introduce buffer agent into the F14 formulation.

### Procedure

Buffer with 3% Trehalose

Approximately 31 mg of trehalose and 0.78 mg of sodium phosphate monobasic monohydrate were added to a vial. 1 mL of F14 was added into the vial, and the solution was vortexed. PSD and osmolality were measured. The vial was sealed and crimped, and stored at −20° C.

Buffer with 5% Trehalose

Approximately 50 mg of trehalose and 0.76 mg of sodium phosphate monobasic monohydrate were added into a vial. 1 mL of F14 was added into the vial, and the solution was vortexed. PSD and osmolality were measured. The vial was sealed and crimped, and stored at −20° C.

### Results

TABLE 11.1
Osmolality* (mOsm/kg) of formulations Prepared

| Name | 3% trehalose | 5% trehalose |
|---|---|---|
| Reading | 85 | 176 |
| Dilution factor | 2 | 2 |
| Osmolality | 170 | 352 |

*Result is corrected with dilution factor (1:1 sample:DI water), Osmolality = Reading × Dilution factor

TABLE 11.2
Particle size distribution of sample with
3% and 5% trehalose after freeze-thaw

| | | Avg | | D(i)10 | D(i)50 | D(i)90 |
|---|---|---|---|---|---|---|
| Sample (%) | Cycles | (nm) | PDI | (nm) | (nm) | (nm) |
| 3% trehalose | 0 | 313.6 | 0.234 | 169 | 373 | 760 |
| | 1 | 348.7 | 0.243 | 186 | 396 | 923 |
| | 2 | 354.1 | 0.274 | 176 | 405 | 905 |
| | 3 | 341.7 | 0.255 | 178 | 382 | 871 |
| 5% trehalose | 0 | 318 | 0.236 | 163 | 376 | 779 |
| | 1 | 341.6 | 0.185 | 212 | 369 | 641 |
| | 2 | 325.5 | 0.255 | 177 | 353 | 720 |
| | 3 | 330.6 | 0.251 | 171 | 370 | 908 |

After 3 cycles of freeze-thaw, both concentrations of trehalose with buffer agents remained the same (no significant change) in particle size.

### Example 7. M2CAB Nano-Suspension Formulation with Ball Milling

Another ball milling batch at 200-mL with higher ratio of surfactants (coded as F15) was made.

TABLE 12.1
Materials

| Name | Grade |
|---|---|
| M2CAB (Prodrug) | N/A |
| Tween 20 | N/A |
| PEG-3350 | N/A |
| Sodium Phosphate | ACS |
| Monobasic | |
| trehalose | NF, EP, JP ChP |
| DI water | N/A |
| HCl | N/A |
| NaOH | N/A |

TABLE 12.2
F15 formulation compounding table for 200 g

| Components | Active | Weight % |
|---|---|---|
| M2CAB (Prodrug) | 79 | 36.4 |
| Tween 20 | 15 | 7.3 |
| PEG-3350 | 15 | 7.3 |
| Sodium phosphate monobasic, monohydrate | 0.176 | |
| trehalose | 11 | 5 |
| water | 99 | 45.2 |
| Total | 220.176 | |

### Procedure-Preparation of Formulation

Approximately 15 g of Tween 20 (polyoxyethylene (20) sorbitan monolaurate) and 15 g of PEG-3350 polymer were added into a glass bottle. 99. g of water was added and the solution was slowly mixed at 700 rpm for 10 min using a magnetic stir bar until all ingredients were completely dissolved.

The solution was then mixed using a high shear mixer at 5,000 rpm, with ice. 10 g of M2CAB powder was transferred into the solution, then the solution was mixed at 8,000 rpm for 2 min until a uniform and flowable suspension formed. The above was repeated and weight transferred, mixing speed, and duration recorded in the table below:

| Intended | Actual Wt | Mixing | | |
|---|---|---|---|---|
| weight (g) | (g) | speed (rpm) | Duration | |
| 10 | 10.006 | 8,000 | 2 | minutes |
| 10 | 10.069 | 8,000 | 2 | minutes |
| 10 | 10.146 | 8,000 | 2 | minutes |
| 10 | 10.066 | 8,000 | 2 | minutes |
| 10 | 10.069 | 8,000 | 5 | minutes |
| 10 | 10.090 | 12,000 | 5 | minutes |
| 10 | 10.079 | 12,000 | 5 | minutes |
| 9 | 9.042 | 12,000 | 10 | minutes |
| Total | 79.567 | N/A | 33 | minutes |

### High-Shear Process

The suspension sample was placed in an ice bath and homogenized with a high shear mixer at high speed (12,000 rpm at 2-8° C. for 15 minutes). An in-process particle size check was performed every 5 min (taking about 5 μL each time), and the results recorded in the table below. The process was stopped if the particle size could not be reduced anymore.

| Shearing time | | | | | |
|---|---|---|---|---|---|
| (min) | Z-avg | PDI | D10 (nm) | D50 (nm) | D90 (nm) |
| 5 | 1750 | 0.174 | 1550 | 1940 | 2440 |
| 10 | 1818 | 0.074 | 1000 | 1200 | 1460 |
| 15 | 2817 | 0.132 | 2130 | 2880 | 2930 |

### Ball-Milling Process

140-150 mL of milling beads (0.4 mm zirconium oxide beads) were added to the grinding tank of the mill using a feed funnel, and the mill moved into the horizontal operating position. The F13 formulation was added to the collecting vessel of the mill, and the overhead stirrer was turned on. The cooling water cocks were opened, and the product pump was turned on starting with the lowest flow rate. As soon as the product exited the outlet into the collecting vessel, the ball mill was started at its lowest speed (10 Hz or 600 rpm) and increased to 2400 rpm in less than a minute.

The F13 formulation was milled at 2400 rpm. The F13 formulation circulation rate was gradually increased while maintaining a pressure of no more than 10, with a record the pump setting of 4. Samples were collected at the time points in the table below, and PSD was measured.

| Time Point | Z-Average | | | | |
|---|---|---|---|---|---|
| (minutes) | (nm) | PDI | D10 (nm) | D50 (nm) | D90 (nm) |
| 2 | 732.7 | 0.434 | 231 | 937 | 1840 |
| 4 | 627.9 | 0.359 | 271 | 565 | 110 |
| 6 | 471.4 | 0.138 | 325 | 486 | 745 |
| 9 | 466.3 | 0.319 | 172 | 635 | 1190 |
| 12 | 398.7 | 0.218 | 224 | 431 | 932 |
| 15 | 368.7 | 0.146 | 262 | 375 | 547 |
| 18 | 387.0 | 0.235 | 214 | 399 | 735 |
| 20 | 355.7 | 0.110 | 241 | 369 | 582 |
| 22 | 350.6 | 0.194 | 213 | 366 | 712 |

### Results

TABLE 12.3
Final Product Results Summary

| Criteria | F15 |
|---|---|
| Appearance | White to off-white suspension |
| Flowability | Free flowing but very viscous |
| pH | 7.94 |
| PSD (nm) | 350.6 |
| PDI | 0.194 |
| HPLC assay (mg/g) | 388.5 |
| HPLC assay (mg/mL) | 377.0 |
| HPLC impurity | No new impurity peak, or |
| | peak growth observed |

### Example 8. Comparison Viscosity and Assays of F13, F14, F15 and F15-Diluted

Viscosity, assay and impurity testing for F13, F14, F15 and F15-diluted was performed.

TABLE 13.1
Materials

| Name | Grade |
|---|---|
| F14 | N/A |
| F13 | N/A |
| F15 | N/A |

### Viscosity Testing Procedure

To test the viscosity: 0.5-mL of each sample was loaded into a cone-plate rheometer container. Equilibrium at 25° C. was reached, and the measurement was taken and the value recorded.

### Results

TABLE 13.2
Assays and impurities of F13, F14, F15 and F15-diluted

| | F14 | | F13 | | | F15 | | | F15-diluted | |
|---|---|---|---|---|---|---|---|---|---|---|
| HPLC assay (mg/g) | 311.0 | 327.9 | 350.7 | 375 | 381.6 | 388.5 | 447.3 | 405.4 | 114.2 | 106.1 |
| % recovery over target conc. | 103% | 109% | 97% | 104% | 106% | 107% | 124% | 113% | N/A | N/A |

| | | | | |
|---|---|---|---|---|
| Initial compounding conc.(mg/g) | 300 | 360 | 360 | 360 |
| HPLC impurity | No | No | No | No |

TABLE 13.3
Viscosity of F13, F14, F15

| Name | F14 | | | F13 | | F15 | | |
|---|---|---|---|---|---|---|---|---|
| Shear rate | 5 | 10 | 5 | 5 | 3 | 1 | 3 | 1 |
| (rpm) | | | | | | | | |
| Viscosity (cP) | 255 | 163.5 | 259 | 591.9 | 838.2 | 2413 | 1052 | 2410 |

### Example 9. Syringe Loading and Injection Force Testing for Formulations

Injection force testing on F14, F13 and F15 was performed.

TABLE 14.1
Materials

| Name | Grade |
|---|---|
| F14 | N/A |
| F13 | N/A |
| F15 | N/A |
| 5-mL syringe | Sterile |
| 3-mL syringe | Sterile |
| 23 G × 1 needle | Sterile |
| 23 G × ½ needle (blunt) | Sterile |
| 25 G × ⅝ needle | Sterile |
| 18 G × 1½ needle | Sterile |

### Procedure

5-mL Syringe with Various Gauge Needles

Two 5-mL syringes were labeled, including the information of formulation code, lot #, syringe size and needle. Each formulation was mixed well and the respective syringe was used to draw up 5 mL formulation with a 18G needle, then the intended needle attached to each.

The sample was taken to a force meter and injection force was measured immediately after loading, with the following parameters: Syringe Inner Diameter (ID) (5-mL is 10.39 mm, 3-mL is 8.19 mm); Sample Volume; Speed=2 mL/min

The injected sample was collected into a 5-mL glass vial, and the injection force (in Newton) for each sample.

### Results

TABLE 14.2
Injection force of F13 (in 5% trehalose), F14
(in water), F15 at 2 mL/min injection rate

| Name | F14 | | F13 | | |
|---|---|---|---|---|---|
| Syringe | 5-mL | 5-mL | 5-mL | 5-mL | 3-mL |
| Needle | 25G ⅝ | 23G × 1 | 23G × 1 | 23G × ½ | 23G × 1 |
| Injection | 50 | 26 | 88 | 59 | 19 |
| force (N) | | | | | |
| Manual | Mild | Mild | High | Mild | Mild |
| injection | to high | resistance | resistance | to high | resistance |
| | resistance | | | resistance | |

In a 5-mL syringe, injection force required was mild for F14 using 23G×1 needle by manual injection. The injection force was measured at 2 mL/min injection rate and the result was close to recommended maximum target of 20 N. Overall, F13 had much higher resistance when injected it through 23G needles in a 5-mL syringe. In a 3-mL syringe 23G×1 needle, injection force required was lower for F13, the value was close to the recommended maximum target of 20 N. 3-mL syringe is recommended for injection of F13.

### Example 10. Prepare F13, F14 and F15 with Buffer Agent and 5% Trehalose for Testing

pH and osmolality testing for F13, F14 and F15 with buffer agent and 5% trehalose were performed.

TABLE 15.1
Materials

| Name | Grade |
|---|---|
| F14 | N/A |
| F13 | N/A |
| F15 | N/A |
| Sodium phosphate monobasic monohydrate | ACS |
| trehalose | NF |

### Procedure-Sample Preparation

F13 Sample with Buffer Agent and 5% Trehalose:
See Example 3 above.
   F14 Sample with Buffer Agent and 5% Trehalose:

Approximately 50 mg of trehalose and 1.1 mg of sodium phosphate monobasic monohydrate were added into a vial. 1 mL of F14 was added into the vial, then the vial was sealed and crimped, and vortexed.

F15 Sample with Buffer Agent and 5% Trehalose

Approximately 51 mg of trehalose and 1.0 mg of sodium phosphate monobasic monohydrate were added into a vial. 1 mL of F15 was added into the vial, then the vial was sealed and crimped, and vortexed.

### Results

TABLE 15.2
pH of F13, F14 and F15

| Criteria | F13 | F14 | F15 |
|---|---|---|---|
| pH | 7.16 | 6.48 | 6.00 |

TABLE 15.3
Osmolality* (mOsm/kg) of formulations Prepared

| Name | F13 | F14 | F15 |
|---|---|---|---|
| Reading | 68 | 176 | 34 |
| Dilution factor | 2 | 2 | 2 |
| Osmolality | 136 | 352 | 68 |

*Result is corrected with dilution factor (1:1 sample: DI water), Osmolality = Reading × Dilution factor

### Example 11. M2CAB Nano-Suspension Formulation with Ball-Milling

TABLE 16.1
Materials and Supplies

| Name | Grade |
|---|---|
| M2CAB (Prodrug) | N/A |
| Tween 20 | N/A |
| PEG-3350 | N/A |
| Sodium Phosphate | ACS |
| Monobasic | |
| trehalose | NF, EP, JP ChP |
| DI water | N/A |

TABLE 16.2
F16 Formulation Compounding Table for 200 g

| Components | Active | Weight % |
|---|---|---|
| M2CAB (Prodrug) | 77 | 35.0 |
| Tween 20 | 11 | 5.0 |
| PEG-3350 | 11 | 5.0 |
| sodium phosphate | 0.176 | 0.1 |
| monobasic, monohydrate | | |
| trehalose | 11 | 5.0 |
| water | 110 | 50.0 |
| Total | 220.176 | |

### Preparation of Formulation F16

11 g of Tween 20 (polyoxyethylene (20) sorbitan monolaurate) And 11 g of PEG-3350 polymer were transferred into a glass bottle. 110 g of water was transferred into the glass vial and the solution was slowly mixed at 700 rpm for 10 minutes with ice, using a magnetic stir bar until all the ingredients were completely dissolved and a clear solution was obtained.

Approximately 10 g M2CAB powder was transferred into the solution, then mixing resumed at 600 rpm for 2 minutes, until a uniform and flowable suspension was formed.

The above steps were repeated and the weight transferred, mixing speed, and duration were recorded as follows:

TABLE 16.3
Weight transferred, mixing speed, and duration

| Intended | Actual | Mixing | |
|---|---|---|---|
| weight (g) | Wt (g) | speed (rpm) | Duration |
| 10 | 10.061 | 600 | 2 minutes |
| 10 | 10.075 | 600 | 2 minutes |
| 10 | 10.084 | 600 | 2 minutes |
| 10 | 10.102 | 600 | 2 minutes |
| 10 | 10.068 | 600 | 2 minutes |
| 10 | 10.024 | 600 | 2 minutes |
| 10 | 10.028 | 600 | 2 minutes |
| 7 | 7.166 | 600 | 2 minutes |
| Total | 77.608 | N/A | 16 minutes |

The solution was slowly mixed at 600 rpm using a propeller stir until M2CAB was completely uniformly dispersed, and the stir process was performed with ice.

### High Shear Process

The suspension sample was placed in an ice bath and homogenized with a Polytron high shear mixer at high speed. An in-process particle size check was performed every 5 min (about 5 μL was taken each time), and results were recorded in the table below. The process was stopped once the particle size could not be reduced anymore. The high shear speed was recorded as 8,000 rpm, the temperature as 2-8° C., and the time as 15 minutes.

TABLE 16.4
In-process Check for High Shear Mixing

| Shearing | | | | | |
|---|---|---|---|---|---|
| time (min) | Z-avg | PDI | D10 (nm) | D50 (nm) | D90 (nm) |
| 5 | 2777 | 0.370 | 936 | 1110 | 1320 |
| 10 | 3358 | 0.245 | 1020 | 1210 | 1420 |
| 15 | 4209 | 0.772 | 599 | 689 | 794 |

### Ball-Milling Process

140-150 mL of milling beads (0.4 mm zirconium oxide beads) were added to the grinding tank of the mill using a feed funnel. The grinding tank was closed and the mill was moved to a horizontal operating position. The F16 formulation was added to the collecting vessel of the mill, and the overhead stirrer was turned on. The cooling water cocks were opened and the product pump was switched on, starting with lowest flow rate.

As soon as the product exited the outlet into the collecting vessel, the mill was started at its lowest speed (10 Hz or 600 rpm) and increased to 2400 rpm in less than a minute. The F16 formulation was milled at 2400 rpm. The F16 formulation circulation rate was gradually increased while maintaining a pressure of no more than 10, with a pump setting of 4.

Samples were collected at the time points in the table below and the particle size distribution (PSD) was measured.

TABLE 16.5
Samples Collected at Different Time Points

| Time Point | | | | | |
|---|---|---|---|---|---|
| (minutes) | Z-avg | PDI | D10 (nm) | D50 (nm) | D90 (nm) |
| 5 | 585.5 | 0.407 | 200 | 744 | 1610 |
| 7 | 510.7 | 0.338 | 180 | 571 | 954 |
| 10 | 424.0 | 0.391 | 179 | 489 | 993 |
| 13 | 400.8 | 0.407 | 168 | 518 | 1030 |
| 15 | 400.1 | 0.398 | 203 | 379 | 666 |
| 18 | 372.7 | 0.373 | 205 | 356 | 594 |

Milling of the F16 formulation continued until the particle size was below 400 nm. The ball-milling sample was collected into a glass bottle, with a final weight of 253.804 g and volume of 145.979 g. The amount of trehalose needed was calculated and the weight of the amount added. The mixture was stirred by overhead mixer. The pH was checked and adjusted as needed to 7.0-7.4 using IN NaOH or HCl.

### Filling Process

Stirring with propeller stir of the bulk suspension was maintained at a speed of 600 rpm. The bulk product was placed into 10-mL vials with a 5.5 mL filling setup for total about 19 units using repeater pump. Each vial was sealed and crimped, and the formulation was stored at 2-8° C.

### Testing

The appearance, pH, osmolality, PSD, and HPLC assay/impurity and uniformity were tested and recorded in the table below (multiple units were tested using 2 mL fill samples).

TABLE 16.6
Final Product Results Summary

| Criteria | F16 |
|---|---|
| Appearance | White to off-white suspension |
| Flowability | Free flowing |
| pH | (7.04 after adding cryo |
| | and buffer agents) |
| Density (g/mL) | 0.9805 (0.9808 after adding |
| | cryo and buffer agents) |
| PSD (nm) | 384 |
| PDI | 0.351 |
| *HPLC assay (mg/g) | 369.2 (342.9 mg/g after |
| | adding cryo and buffer agents) |
| HPLC assay (mg/mL) | 362.0 (336.2 mg/mL after |
| | adding cryo and buffer agents) |
| HPLC impurity | ND |

*Average of four samples

TABLE 16.7
Content of Uniformity

| Exemplary | HPLC | HPLC |
|---|---|---|
| Samples | assay (mg/g) | assay (mg/mL) |
| (#2) | 365.3 | 358.2 |
| (#3) | 372.0 | 364.8 |
| (#4) | 371.7 | 364.5 |
| (#6) | 367.7 | 360.5 |

### Example 12. Syringe Loading and Injection Force Testing for F16

Purpose: To perform an injection force testing on F16.

TABLE 17.1
Materials

| Name | Grade |
|---|---|
| F16 | N/A |
| 5-mL syringe | Sterile |
| 18 G × 1½″ needle | Sterile |
| 21 G × 1½″ needle | Sterile |
| 22 G × 1″ needle | Sterile |
| 22 G × 1½″ needle | Sterile |
| 23 G × 1″ needle | Sterile |
| 23 G × 1½″ needle | Sterile |
| 25 G × 1½″ needle | Sterile |

### Procedure

5-mL syringe with various gauge needles: two 5-mL syringes were labeled to include the information of formulation code, lot number, syringe size and needle. Each formulation was mixed well and the respective syringe was used to draw up 5 mL formulation, then connected to a needle. The size of the needle was recorded.

The sample was measured on a force meter immediately after loading by placing the loaded syringe on the force meter and starting the force meter with the following parameters: Syringe Inner Diameter (ID) (5-mL is 10.39 mm), Corresponding Sample Volume, and Speed=5 mL/min.

The injected sample was collected in a 5-mL glass vial. The above steps were repeated using different needle sizes to test the syringe force for formulation.

### Results

TABLE 17.2
Injection force of F16 (5% trehalose and buffer agent containing)
and various dilutions (Syringe-5 mL, Flow rate-5 mL/min)

| Name/Needle | | 21G × 1.5 | 22G × 1 | 22G × 1.5 | 23G × 1 | 23G × 1.5 | 25G × 1.5 |
|---|---|---|---|---|---|---|---|
| F16 (343 mg/g) | Manual | Mild to | Mild | Moderate | High | High | NT |
| 336 mg/mL | Injection | moderate | resistance | resistance | resistance | resistance | |
| | | resistance | | | | | |
| | Injection | 19.1 | 16 | 22.1 | 29 | 44 | NT |
| | Force | | | | | | |
| F16 (320 mg/g) | Manual | NT | NT | Mild | Moderate | Moderate | NT |
| 314 mg/mL | Injection | | | resistance | resistance | to high | |
| | | | | | | resistance | |
| | Injection | NT | NT | 16 | 21 | 27 | NT |
| | Force | | | | | | |
| F16 (300 mg/g) | Manual | NT | NT | NT | NT | Moderate | High |
| 293 mg/mL | Injection | | | | | resistance | resistance |
| | Injection | NT | NT | NT | NT | 20 | 40 |
| | Force | | | | | | |

* Decision to use original F16 formulation, with 22G 1.5-inch needle.

### Example 13. DP Process to Make M2CAB Drug Product F16 Formulation

TABLE 20.1
Materials and Supplies

| Name | Grade |
|---|---|
| M2CAB (Prodrug) | N/A |
| Tween 20 | N/A |
| PEG-3350 | N/A |
| Sodium phosphate | ACS |
| monobasic, monohydrate | |
| Sodium phosphate dibasic, | N/A |
| heptahydrate | |
| trehalose | N/A |
| Sodium Hydroxide | N/A |
| Hydrochloric acid | N/A |

TABLE 20.2
F16 Formulation Compounding

| Components | Active (g) | Weight % | w/v % |
|---|---|---|---|
| M2CAB (Prodrug) | 100.3 | 35 | 34.3 |
| Tween 20 | 14.3 | 5.0 | 4.9 |
| PEG-3350 | 14.3 | 5.0 | 4.9 |
| Sodium phosphate | 0.190 (eq 1.38 | 0.1 | 0.1 |
| monobasic, monohydrate | mmol) | | |
| Sodium phosphate | 0.424 (eq 1.58 | 0.1 | 0.1 |
| dibasic, heptahydrate | mmol) | | |
| trehalose | 14.3 | 5.0 | 4.9 |
| water | 143 | 49.9 | QS |
| Total | 286.8 | | 292.7 |

### Preparation of Formulation

Approximately 14 g of Tween 20 (Polyoxyethylene (20) sorbitan monolaurate) and 14 g of PEG-3350 polymer were added into a glass bottle. 143 g of water was added to the glass vial.

The solution was slowly mixed at 600 rpm using a magnetic stir bar until all ingredients were completely dissolved (about 15 minutes). Once a clear solution was obtained, the stirring process was stopped.

The solution was slowly mixed again at 600 rpm with a propeller stirrer, with ice. 10 g of M2CAB powder was added into the solution. The mixer was reinserted and mixing was resumed at 600 rpm for 2 minutes until a uniform and flowable suspension formed.

The above steps were repeated and the weight transferred, mixing
speed, and duration were recorded in the table below:

| Intended | Actual | Mixing | |
|---|---|---|---|
| weight (g) | Wt (g) | speed (rpm) | Duration |
| 10 | 10.2183 | 600 | 2 minutes |
| 10 | 10.0287 | 600 | 2 minutes |
| 10 | 10.0303 | 600 | 2 minutes |
| 10 | 10.1320 | 600 | 2 minutes |
| 10 | 10.0888 | 600 | 2 minutes |
| 10 | 10.1359 | 600 | 10 minutes |
| 10 | 10.0283 | 600 | 2 minutes |
| 10 | 10.0237 | 600 | 10 minutes |
| 10 | 10.0456 | 600 | 2 minutes |
| 10.3 | 10.4025 | 600 | 2 minutes |

The solution was slowly mixed using a propeller stir at 600 rpm, with ice, until M2CAB was completely uniformly dispersed (between 12-24 hours).

### High Shear Process

TABLE 20.3
High Shear Process

| Shearing | | | | | |
|---|---|---|---|---|---|
| time (min) | Z-avg | PDI | D10 (nm) | D50 (nm) | D90 (nm) |
| 5 | 3527 | 0.547 | 891 | 1040 | 1200 |
| 10 | 2522 | 0.518 | 1860 | 2440 | 3200 |
| 15 | 0.518 | 0.112 | 2000 | 3000 | 4640 |

### Ball-Milling Process

140-150 mL of milling beads (0.4 mm zirconium oxide beads) were added to the grinding tank of the mill using a feed funnel. The grinding tank was closed and the mill was moved into the horizontal operating position. The F16 formulation was added to the collecting vessel of the mill, and the overhead stirrer was turned on. The cooling water cocks were opened and the product pump was switched on, starting with the lowest flow rate.

As soon as the product exited the outlet into the collecting vessel, the mill was started at its lowest speed (10 Hz or 600 rpm) and increased to 2400 rpm in less than a minute. The F16 formulation was milled at 2400 rpm. The F16 formulation circulation rate was gradually increased while maintaining a pressure of no more than 10 and a pump setting of 4. Samples were collected at the time points in the table below, and the PSD was measured.

| Time Point | | | | | |
|---|---|---|---|---|---|
| (minutes) | Z-Average | PDI | D10 (nm) | D50 (nm) | D90 (nm) |
| 2 | 848.4 | 0.037 | 615 | 884 | 1270 |
| 6 | 487.3 | 0.194 | 337 | 472 | 685 |
| 9 | 439.3 | 0.322 | 245 | 425 | 2600 |
| 12 | 405.8 | 0.161 | 276 | 409 | 614 |
| 15 | 394.4 | 0.374 | 203 | 371 | 866 |
| 18 | 374.0 | 0.253 | 199 | 395 | 863 |
| 21 | 407.8 | 0.226 | 261 | 388 | 616 |

### Filling Process

Stirring or mixing of the bulk suspension was continued, then the bulk product was added to about 30-36 vials with 6 mL filling using a manual/repeater pump. Each vial was crimp sealed, and stored at 2-8° C.

### Testing

The appearance, pH, osmolality, particle size distribution (PSD), and HPLC assay/impurity and uniformity were tested (test multiple units using 6 mL fill samples), and filled vials were sent for gamma sterilization.

### Results

TABLE 20.4
Final Product Results Summary

| Criteria | F16 |
|---|---|
| Appearance | White to off-white suspension |
| Flowability | Free flowing |
| pH | 7.08 |
| Density (g/mL) | TBD |
| PSD (nm) | 387.6 |
| PDI | 0.331 |
| *HPLC assay (mg/g) | TBD |
| HPLC assay (mg/mL) | TBD |
| HPLC impurity | TBD |

### Example 14. Pre-Gamma and Post-Gamma Irradiation of F16

pre- and post-gamma irradiated F16 formulations were compared. The target gamma-ray dose was 25 kGy, and the actual dose delivered was between 27.9 kGy to 28.5 kGy with a total exposure time of 581 minutes.

### Results

TABLE 21.1
Results of Pre-gamma and Post-gamma F16 Formulations

| Criteria | Pre-gamma F16 | Post-gamma F16 |
|---|---|---|
| Appearance | White to off-white | Off-white |
| | suspension | suspension |
| Flowability | Free flowing | Free flowing |
| pH | 7.04 | 6.88 |
| Density (g/mL) | 0.9805 | NT |
| PSD (nm) | 384 | 369.5 |
| PDI | 0.351 | 0.272 |
| *HPLC assay (mg/g) | 342.9 | 346.0 |
| HPLC assay (mg/mL) | 336.2 | 339.1 |
| HPLC impurity | ND | ND |

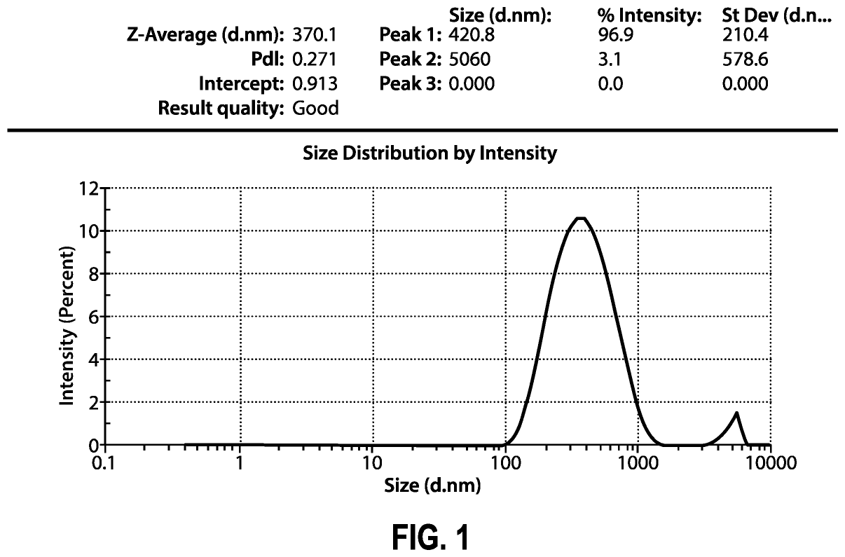

FIG. 1 depicts a histogram of particle size distribution (PSD) of post-gamma radiation-treated F16.

### Example 15. Stability of F16 at 40° C.

Stability measurements at accelerated conditions (40° C.) of F16 formulations were performed over a 41 day period.

### Results

TABLE 22.1
Appearances, Flowability, PSD, and pH of F16 formulations at various time points.

| | Storage | | | | | | |
|---|---|---|---|---|---|---|---|
| Name | (° C.) | Test | T0 | At 6 days | At 17 days | At 24 days | At 41 days |
| F16 | 40° C. | Appearances | White to | White to | White to | White to | Off White |
| | | | Of White | Of White | Of White | Of White | to Light |
| | | | | | | | Tan |
| | | Flowability | Free flow | Free flow | Free flow | Free flow | Free flow |
| | | pH | 7.04 | NT | 7.03 | 6.96 | 7.13 |
| | | Z-avg (nm) | 384 | 403.8 | 413.6 | 414.9 | 422.3 |
| | | PDI | 0.351 | 0.213 | 0.277 | 0.251 | 0.223 |
| | | D(i)10 (nm) | 188 | 247 | 222 | 237 | 249 |
| | | D(i)50 (nm) | 406 | 416 | 462 | 419 | 439 |
| | | D(i)90 (nm) | 1010 | 1010 | 778 | 1120 | 1040 |

### Example 16. Exemplary Procedure for Production of M2CAB Formulation F16

This is a procedure for the preparation of Formulation F16. Tween 20 (Polyoxyethylene (20) sorbitan monolaurate) is added into a first container. A sterile scoop is used to transfer PEG3350 into the container is then recorded. Water for injection (WFI) is then added to the container. The stir bar is next added into the first container, Stirring is then performed at 600-800 rpm for approximately 10 minutes until a visual clear solution is obtained The stir bar is then removed. The container is placed into an ice bath. Using an overhead mixer at 500-700 rpm, the solution is slowly mixed. The total weight of M2CAB into the first container is transferred stepwise with about of the 10-20% of total weight during each step, and the sample is stirred while forming a well dispersed suspension each time before adding more M2CAB. The mixing with the overhead mixer at 500-700 rpm in the first container is continued for about 1-2 hour until a uniform suspension is formed. The rpm, temperature, and end time is recorded. The container is kept in the ice bath and Silverson high shear mixer is used to mix the suspension in the Container at 8,000+/−500 rpm for about 15-30 minutes to further disperse the formulation.

The initial cycle of ball milling is started, where a procedure is as follows: a. Cool down the ball mill system to 5° C. b. Insert overhead mixer into the first container and stir the mixture at 400+/−40 rpm to prevent suspension from settling. c. If necessary, scrape the bottom of the first container with a spatula to resuspend any large particles. d. Insert the inlet tubing of ball mill into the first container and outlet tubing into a second container. e. A peristaltic pump is turned on with minimal flow rate and let the suspension flow to the milling chamber. f. A ball-mill rotor at minimal speed (10 Hz or 600+/−60 rpm) is turned on with the speed to 2,400+/−240 rpm within 2 minutes. g. The flow rate of the pump is gradually increased to ensure the pressure of milling chamber is below 15 PSI. h. When all the suspension has passed the milling chamber, the pump is stopped, and about 0.2 mL aliquot sample using sterile pipette is taken. j. The particle size distribution of the collected sample is measured. The process is continued until the average particle size of milling product is below 400 nm.

Ball milling cycles are continued to reduce the particle size, where a procedure is as follows: a. The second container is gently shaken. The solution in the then poured into the first container. The outlet tubing is placed back into the second container. b. The peristaltic pump is turned on and the flow rate is gradually increased to ensure the pressure of milling chamber is below 15 PSI. c. After each cycle, a 1 mL aliquot sample is collected using a sterile pipette for particle size distribution (PSD) measurement. f. The steps are repeated until the average particle size of milling product is below 400 nm.

The nanosuspension is kept stirring in the second container by an overhead mixer for the remainder of the process in a vortex with minimum foaming. A sterile scoop is used to transfer trehalose into a weigh dish an which is then transferred into the second container. A sterile scoop is used to transfer sodium phosphate monobasic monohydrate into a weigh dish which is then transferred into the second container. A sterile scoop is used to transfer sodium phosphate dibasic heptahydrate into a weigh dish which is transferred into the second container. If needed, pH is adjusted to 7.0±0.2 by adding IN NaOH/HCl. Acceptable pH limits are 6.8 to 7.2. The stirring of the nanosuspension in the second container is stopped and end time is recorded. The overhead mixer is inserted into the nanosuspension, with stirring to be kept at 600-800 rpm. The stirring speed and start time is recorded. The stir speed is to be adjusted to generate a vortex without excessive foaming. The stirring of the suspension by overhead mixer is maintained. The final product is then filled into a 10-mL type 1 glass vial by a repeater pump. After vials are filled, each vial with 18 mm sterile stopper are closed immediately inside a biosafety cabinet. Each vial with a 20 mm cap are crimp sealed. Vials are placed in each of the secondary cartons. The carton is kept at 2-8° C. and the storage time is recorded.

### Example 17. Crystalline Forms of M2CAB in Compositions

Crystalline forms of M2CAB in compositions of the present disclosure are provided. The M2CAB present in the compositions (e.g., Formulations F13-F16) are characterized by a mixture of crystalline forms of M2CAB (Forms 1+4), which have a powder x-ray diffraction pattern as shown in FIG. 2. Exemplary peak listings are given in Table 23 below.

XRPD patterns were obtained using a T2 high-throughput XRPD set-up. The plates were mounted on a Bruker General Area Detector Diffraction System (GADDS) equipped with a VÅNTEC-500 gas area detector corrected for intensity and geometric variations. The calibration of the measurement accuracy (peaks position) was performed using NIST SRM1976 standard (Corundum).

Data collection was carried out at room temperature using monochromatic Cu Kα radiation in the 20 region between 1.5° and 41.5°, which is the most distinctive part of the XRPD pattern. The diffraction pattern of each well was collected in two 2θ ranges (1.5°≤2θ≤21.5° for the first frame, and 19.5°≤2θ≤41.5° for the second) with an exposure time of 90 seconds for each frame. No background subtraction or curve smoothing was applied to the XRPD patterns.

TABLE 23
Peak listings for Form 1 + 4.

| Angle 2-Theta ° | Intensity % |
|---|---|
| 3.382 | 100 |
| 6.761 | 5.2 |
| 6.976 | 5.3 |
| 7.177 | 2.5 |
| 7.403 | 3.7 |
| 10.164 | 0.6 |
| 10.387 | 0.4 |
| 11.209 | 1.6 |
| 13.955 | 1.1 |
| 14.361 | 0.6 |
| 14.859 | 0.6 |
| 15.428 | 0.6 |
| 15.694 | 0.8 |
| 16.342 | 1.8 |
| 16.614 | 2.2 |
| 16.814 | 2.3 |
| 17.566 | 3.1 |
| 17.82 | 3 |
| 18.201 | 1.1 |
| 18.718 | 2.2 |
| 19.339 | 1.1 |
| 19.923 | 2.4 |
| 20.651 | 1.1 |
| 21.036 | 2.5 |
| 21.598 | 8.1 |
| 21.886 | 4 |
| 22.152 | 2.4 |
| 22.678 | 2.6 |
| 22.852 | 4.1 |
| 23.25 | 1.5 |
| 23.472 | 1.9 |
| 23.632 | 3 |
| 24.02 | 0.9 |
| 24.284 | 2.2 |
| 24.542 | 0.6 |
| 24.724 | 1 |
| 25.041 | 0.7 |
| 25.597 | 1 |
| 26.122 | 1.8 |
| 27.065 | 1.2 |
| 28.297 | 0.6 |
| 28.986 | 0.9 |
| 29.359 | 0.3 |
| 29.882 | 0.4 |
| 30.121 | 0.5 |
| 30.882 | 0.3 |
| 31.72 | 0.5 |
| 33.012 | 0.2 |
| 33.35 | 0.3 |

What is claimed is:

1. A pharmaceutical composition comprising:
(i) a crystalline form of a compound represented by: 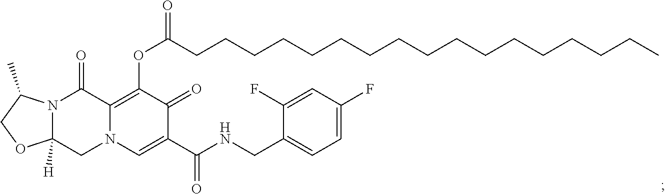 [SMILES: CCCCCCCCCCCCCCCCCC(=O)Oc1c2n(cc(C(=O)NCc3ccc(F)cc3F)c1=O)C[C@H]1OC[C@H](C)N1C2=O]
and
(ii) a cryoprotectant.

2. The pharmaceutical composition of claim 1, wherein the cryoprotectant is a sugar.

3. The pharmaceutical composition of claim 2, wherein the cryoprotectant is selected from the group consisting of trehalose, sucrose, mannitol, and any combination thereof.

4. The pharmaceutical composition of any one of claims 1-3, wherein the cryoprotectant is trehalose.

5. The pharmaceutical composition of any one of claims 1-4, comprising about 3 weight percent to about 10 weight percent of the cryoprotectant, based on the total weight of the pharmaceutical composition.

6. The pharmaceutical composition of any one of claims 1-5, comprising about 5 weight percent of the cryoprotectant, based on the total weight of the pharmaceutical composition.

7. The pharmaceutical composition of any one of claims 1-6, comprising a surfactant.

8. The pharmaceutical composition of claim 7, comprising about 3 weight percent to about 5 weight percent of the surfactant, based on the total weight of the pharmaceutical composition.

9. The pharmaceutical composition of any one of claims 1-8, comprising a polyethylene glycol.

10. The pharmaceutical composition of claim 9, comprising about 3 weight percent to about 5 weight percent of the polyethylene glycol, based on the total weight of the pharmaceutical composition.

11. The pharmaceutical composition of any one of claims 1-10, comprising a phosphate buffer.

12. The pharmaceutical composition of any one of claims 1-11, wherein the pH of the composition is from about 6.0 to about 7.5.

13. The pharmaceutical composition of any one of claims 1-12, wherein the pH of the composition is about 7.0.

14. The pharmaceutical composition of any one of claims 1-13, comprising about 25 weight percent to about 45 weight percent of the crystalline form, based on the total weight of the pharmaceutical composition.

15. The pharmaceutical composition of any one of claims 1-14, comprising a plurality of nanoparticles comprising the crystalline form of the compound.

16. The pharmaceutical composition of any one of claims 7-15, comprising a plurality of nanoparticles comprising the crystalline form of the compound and the surfactant.

17. The pharmaceutical composition of any one of claims 7-16, comprising a plurality of nanoparticles comprising the crystalline form of the compound and the polyethylene glycol.

18. The pharmaceutical composition of any one of claims 7-17, comprising a plurality of nanoparticles comprising the crystalline form of the compound, the surfactant, and the polyethylene glycol.

19. The pharmaceutical composition of any one of claims 15-18, wherein the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm.

20. The pharmaceutical composition of any one of claims 15-19, wherein the plurality of nanoparticles has a z-average particle size of 200 nm to 900 nm.

21. The pharmaceutical composition of any one of claims 15-20, wherein the plurality of nanoparticles has a z-average particle size of 275 nm to 425 nm.

22. The pharmaceutical composition of any one of claims 15-21, wherein the plurality of nanoparticles has a polydispersity index of about 0.15 to about 0.4.

23. The pharmaceutical composition of any one of claims 15-22, wherein the plurality of nanoparticles a polydispersity index of about 0.25 to about 0.35.

24. A pharmaceutical composition comprising:
(i) a mixture of crystalline forms of a compound represented by: 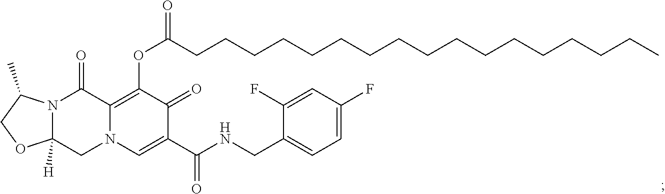 [SMILES: CCCCCCCCCCCCCCCCCC(=O)Oc1c2n(cc(C(=O)NCc3ccc(F)cc3F)c1=O)C[C@H]1OC[C@H](C)N1C2=O]
and
(ii) a cryoprotectant.

25. The pharmaceutical composition of claim 24, wherein the cryoprotectant is a sugar.

26. The pharmaceutical composition of claim 24 or 25, wherein the cryoprotectant is selected from the group consisting of trehalose, sucrose, mannitol, and any combination thereof.

27. The pharmaceutical composition of any one of claims 24-26, wherein the cryoprotectant is selected from the group consisting of trehalose, sucrose, and mannitol.

28. The pharmaceutical composition of any one of claims 24-27, wherein the cryoprotectant is trehalose.

29. The pharmaceutical composition of any one of claims 24-28, comprising about 3 weight percent to about 10 weight percent of the cryoprotectant, based on the total weight of the pharmaceutical composition.

30. The pharmaceutical composition of any one of claims 24-29, comprising about 5 weight percent of the cryoprotectant, based on the total weight of the pharmaceutical composition.

31. The pharmaceutical composition of any one of claims 24-30, comprising a surfactant.

32. The pharmaceutical composition of claim 31, comprising about 3 weight percent to about 5 weight percent of the surfactant, based on the total weight of the pharmaceutical composition.

33. The pharmaceutical composition of any one of claims 24-32, comprising a polyethylene glycol.

34. The pharmaceutical composition of claim 33, comprising about 3 weight percent to about 5 weight percent of the polyethylene glycol, based on the total weight of the pharmaceutical composition.

35. The pharmaceutical composition of any one of claims 24-34, comprising a phosphate buffer.

36. The pharmaceutical composition of any one of claims 24-35, wherein the pH of the composition is from about 6.0 to about 7.5.

37. The pharmaceutical composition of any one of claims 24-36, wherein the pH of the composition is about 7.0.

38. The pharmaceutical composition of any one of claims 24-37, wherein the mixture of crystalline forms of the compound has two crystalline forms.

39. The pharmaceutical composition of any one of claims 24-38, wherein one of the two crystalline forms is present in about 10 to 30 weight percent of the mixture and the other crystalline form in about 70 to 90 weight percent of the mixture.

40. The pharmaceutical composition of any one of claims 24-39, wherein the mixture of crystalline forms of the compound is characterized by a powder X-ray diffraction pattern having a characteristic peak in degrees 2θ at about 21.6.

41. The pharmaceutical composition of any one of claims 24-40, wherein the mixture of crystalline forms of the compound is characterized by a powder X-ray diffraction pattern having a characteristic peak in degrees 2θ at about 7.0, 17.8, and 21.6.

42. The pharmaceutical composition of any one of claims 24-41, wherein the mixture of crystalline forms of the compound is characterized by a powder X-ray diffraction pattern having a characteristic peak in degrees 2θ at about 7.0, 7.4, 17.8, 21.6, and 23.6.

43. The pharmaceutical composition of any one of claims 24-42, wherein the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1.

44. The pharmaceutical composition of any one of claims 24-43, wherein the mixture of crystalline forms of the compound is characterized by an XRPD pattern substantially as depicted in FIG. 2.

45. The pharmaceutical composition of any one of claims 24-44, comprising about 25 weight percent to about 45 weight percent of the mixture of crystalline forms of the compound, based on the total weight of the pharmaceutical composition.

46. The pharmaceutical composition of any one of claims 24-45, comprising a plurality of nanoparticles comprising the mixture of crystalline forms of the compound.

47. The pharmaceutical composition of any one of claims 31-46, comprising a plurality of nanoparticles comprising the mixture of crystalline forms of the compound and the surfactant.

48. The pharmaceutical composition of any one of claims 31-47, comprising a plurality of nanoparticles comprising the mixture of crystalline forms of the compound and the polyethylene glycol.

49. The pharmaceutical composition of any one of claims 31-48, comprising a plurality of nanoparticles comprising the mixture of crystalline forms of the compound, the surfactant, and the polyethylene glycol.

50. The pharmaceutical composition of any one of claims 46-49, wherein the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm.

51. The pharmaceutical composition of any one of claims 46-50, wherein the plurality of nanoparticles has a z-average particle size of 200 nm to 900 nm.

52. The pharmaceutical composition of any one of claims 46-51, wherein the plurality of nanoparticles has a z-average particle size of 275 nm to 425 nm.

53. The pharmaceutical composition of any one of claims 46-52, wherein the plurality of nanoparticles has a polydispersity index of about 0.15 to about 0.4.

54. The pharmaceutical composition of any one of claims 46-53, wherein the plurality of nanoparticles has a polydispersity index of about 0.25 to about 0.35.

55. A method of treating, inhibiting, and/or preventing a viral infection in a patient in need thereof, comprising administering to the patient an effective amount of the pharmaceutical composition of any one of claims 1-54.

56. The method of claim 55, wherein the viral infection is a retroviral infection.

57. The method of claim 55 or 56, wherein the viral infection is an HIV infection.

58. The method of any one of claims 55-57, comprising administering the pharmaceutical composition intramuscularly to the individual.

59. A process for preparing a pharmaceutical composition comprising:
(i) a crystalline form of a compound represented by: 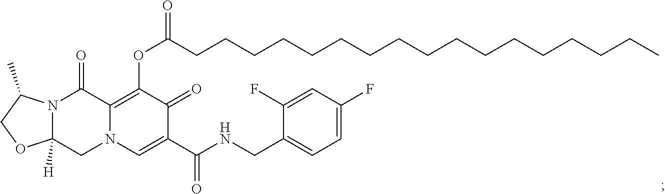 [SMILES: CCCCCCCCCCCCCCCCCC(=O)Oc1c2n(cc(C(=O)NCc3ccc(F)cc3F)c1=O)C[C@H]1OC[C@H](C)N1C2=O]
and
(ii) a cryoprotectant;
the process comprising:
(a) providing a suspension comprising the crystalline form of the compound;
(b) mixing the suspension using a ball milling rotor, thereby preparing a ball-milled mixture; and
(c) combining the ball-milled mixture with the cryoprotectant,
thereby preparing the pharmaceutical composition.

60. The process of claim 59, wherein the suspension comprises one or more excipients selected from the group consisting of a polyethylene glycol, a surfactant, and combinations thereof.

61. The process of claim 59 or 60, wherein the cryoprotectant is a sugar.

62. The process of any one of claims 59-61, wherein the cryoprotectant is selected from the group consisting of trehalose, sucrose, mannitol, and any combination thereof.

63. The process of any one of claims 59-62, wherein the cryoprotectant is trehalose.

64. The process of any one of claims 59-63, wherein the pharmaceutical composition comprises about 3 weight percent to about 10 weight percent of the cryoprotectant, based on the total weight of the pharmaceutical composition.

65. The process of any one of claims 59-64, wherein the pharmaceutical composition comprises about 5 weight percent of the cryoprotectant, based on the total weight of the pharmaceutical composition.

66. The process of any one of claims 51-65, wherein the pharmaceutical composition comprises a surfactant.

67. The process of any one of claim 66, wherein the pharmaceutical composition comprises about 3 weight percent to about 5 weight percent of the surfactant, based on the total weight of the pharmaceutical composition.

68. The process of any one of claims 51-59, wherein the pharmaceutical composition comprises a polyethylene glycol.

69. The process of claim 68, wherein the pharmaceutical composition comprises about 3 weight percent to about 5 weight percent of the polyethylene glycol, based on the total weight of the pharmaceutical composition.

70. The process of any one of claims 59-69, combining the ball-milled mixture with the cryoprotectant with a phosphate buffer.

71. The process of any one of claims 59-70, wherein the pH of the composition is from about 6.0 to about 7.5.

72. The process of any one of claims 59-71, wherein the pH of the composition is about 7.0.

73. The process of any one of claims 59-72, wherein the pharmaceutical composition comprises about 25 weight percent to about 45 weight percent of the crystalline form, based on the total weight of the pharmaceutical composition.

74. The process of any one of claims 59-73, wherein the pharmaceutical composition comprises a plurality of nanoparticles comprising the crystalline form of the compound.

75. The process of any one of claims 66-74, wherein the pharmaceutical composition comprises a plurality of nanoparticles comprising the crystalline form of the compound and the surfactant.

76. The process of any one of claims 66-75, wherein the pharmaceutical composition comprises a plurality of nanoparticles comprising the crystalline form of the compound and the polyethylene glycol.

77. The pharmaceutical composition of any one of claims 66-76, wherein the pharmaceutical composition comprises a plurality of nanoparticles comprising the crystalline form of the compound, the surfactant, and the polyethylene glycol.

78. The process of any one of claims 74-77, wherein the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm.

79. The process of any one of claims 74-78, wherein the plurality of nanoparticles has a z-average particle size of 200 nm to 900 nm.

80. The process of any one of claims 74-79, wherein the plurality of nanoparticles has a z-average particle size of 275 nm to 425 nm.

81. The process any one of claims 74-80, wherein the plurality of nanoparticles has a polydispersity index of about 0.15 to about 0.4.

82. The process of any one of claims 74-81, wherein plurality of nanoparticles in the pharmaceutical composition has a polydispersity index of about 0.25 to about 0.35.

83. A process for preparing a pharmaceutical composition comprising:
(i) a mixture of crystalline forms of a compound represented by: 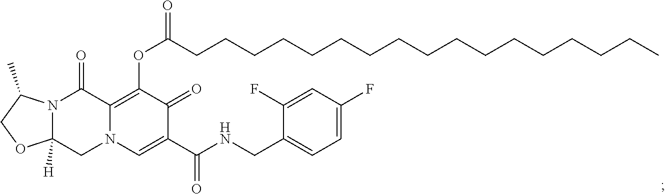 [SMILES: CCCCCCCCCCCCCCCCCC(=O)Oc1c2n(cc(C(=O)NCc3ccc(F)cc3F)c1=O)C[C@H]1OC[C@H](C)N1C2=O]
and
(ii) a cryoprotectant;
the process comprising:
(a) providing a suspension comprising the mixture of crystalline forms of the compound;
(b) mixing the suspension using a ball milling rotor, thereby preparing a ball-milled mixture; and
(c) combining the ball-milled mixture with the cryoprotectant,
thereby preparing the pharmaceutical composition.

84. The process of claim 83, wherein the cryoprotectant is a sugar.

85. The process of claim 83 or 84, wherein the cryoprotectant is selected from the group consisting of trehalose, sucrose, mannitol, and any combination thereof.

86. The process of any one of claims 83-85, wherein the cryoprotectant is selected from the group consisting of trehalose, sucrose, and mannitol.

87. The pharmaceutical composition of any one of claims 83-86, wherein the cryoprotectant is trehalose.

88. The process of any one of claims 83-87, wherein the pharmaceutical composition comprises about 3 weight percent to about 10 weight percent of the cryoprotectant, based on the total weight of the pharmaceutical composition.

89. The process of any one of claims 83-88, wherein the pharmaceutical composition comprises about 5 weight percent of the cryoprotectant, based on the total weight of the pharmaceutical composition.

90. The process of any one of claims 83-89, wherein the pharmaceutical composition comprises a surfactant.

91. The process of claim 90, wherein the pharmaceutical composition comprises about 3 weight percent to about 5 weight percent of the surfactant, based on the total weight of the pharmaceutical composition.

92. The process of any one of claims 83-91, wherein the pharmaceutical composition comprises a polyethylene glycol.

93. The process of claim 92, wherein the pharmaceutical composition comprises about 3 weight percent to about 5 weight percent of the polyethylene glycol, based on the total weight of the pharmaceutical composition.

94. The process of any one of claims 83-93, wherein the pharmaceutical composition comprises a phosphate buffer.

95. The process of any one of claims 83-94, wherein the pH of the composition is from about 6.0 to about 7.5.

96. The process of any one of claims 83-95, wherein the pH of the composition is about 7.0.

97. The process of any one of claims 83-96, wherein the mixture of crystalline forms of the compound has two crystalline forms.

98. The process of any one of claims 83-97, wherein one of the two crystalline forms is present in about 10 to 30 weight percent of the mixture and the other crystalline form is about 70 to 90 weight percent of the mixture.

99. The process of any one of claims 83-98, wherein the mixture of crystalline forms of the compound is characterized by a powder X-ray diffraction pattern having a characteristic peak in degrees 2θ at about 21.6.

100. The process of any one of claims 83-99, wherein the mixture of crystalline forms of the compound is characterized by a powder X-ray diffraction pattern having a characteristic peak in degrees 2θ at about 7.0, 17.8, and 21.6.

101. The process of any one of claims 83-100, wherein the mixture of crystalline forms of the compound is characterized by a powder X-ray diffraction pattern having a characteristic peak in degrees 2θ at about 7.0, 7.4, 17.8, 21.6, and 23.6.

102. The process of any one of claims 83-101, wherein the mixture of crystalline forms of the compound is characterized by a powder X-ray diffraction pattern having a characteristic peak in degrees 2θ at about 7.0, 7.4, 14.0, 16.3, 16.8, 17.8, 21.6, 23.6, 24.3, and 26.1.

103. The process of any one of claims 83-102, wherein the mixture of crystalline forms of the compound is characterized by an XRPD pattern substantially as depicted in FIG. 2.

104. The process of any one of claims 83-103, comprising about 25 weight percent to about 45 weight percent of the mixture of crystalline forms of the compound, based on the total weight of the pharmaceutical composition.

105. The process of any one of claims 83-104, wherein the pharmaceutical composition comprises a plurality of nanoparticles comprising the mixture of crystalline forms of the compound.

106. The process of any one of claims 83-105, wherein the pharmaceutical composition comprises a plurality of nanoparticles comprising the mixture of crystalline forms of the compound and the surfactant.

107. The process of any one of claims 90-106, wherein the pharmaceutical composition comprises a plurality of nanoparticles comprising the mixture of crystalline forms of the compound and the polyethylene glycol.

108. The process of any one of claims 90-107, wherein the pharmaceutical composition comprises a plurality of nanoparticles comprising the mixture of crystalline forms of the compound, the surfactant, and the polyethylene glycol.

109. The process of any one of claims 90-108, wherein the plurality of nanoparticles has a particle size distribution (D10) of about 250 nm to about 650 nm, a particle size distribution (D50) of about 350 nm to about 900 nm, and a particle size distribution (D90) of about 600 nm to about 1300 nm.

110. The process of any one of claims 105-109, wherein the plurality of nanoparticles has a z-average particle size of 200 nm to 900 nm.

111. The process of any one of claims 105-110, wherein the plurality of nanoparticles has a z-average particle size of 275 nm to 425 nm.

112. The process of any one of claims 105-111, wherein the plurality of nanoparticles has a polydispersity index of about 0.15 to about 0.4.

113. The process of any one of claims 105-112, wherein the plurality of nanoparticles has a polydispersity index of about 0.25 to about 0.35.
